(19) United States
(12) Patent Application Publication
(10) Pub. No.: US 2007/0289605 A1
(43) Pub. Date: Dec. 20, 2007

(54) Use of dilute hydrogen peroxide to remove DNA contamination

(75) Inventors: Ashlee Salloom, Nottingham, MD (US); Matt Collis, Seven Valleys, PA (US)

Correspondence Address: DAVID W. HIGHET, VP & CHIEF INTELLECTUAL PROPERTY;COUNSEL BECTON, DICKINSON AND COMPANY, BIRCH, STEWART, KOLASCH & BIRCH, LLP, 1 BECTON DRIVE, MC 110, FRANKLIN LAKES, NJ, 07417-1880, US

(73) Assignee: BECTON, DICKINSON AND COMPANY, Franklin Lakes, NJ (US)

(21) Appl. No.: 11/543,781

(22) Filed: Oct. 6, 2006

Related U.S. Application Data
(60) Provisional application No. 60/724,302, filed on Oct. 7, 2005.

(51) Int. Cl. A61L 2/00

(52) U.S. Cl. 134/6; 422/28

(57) ABSTRACT

The present invention provides a method of reducing nucleic acid contamination on a surface, which comprises the steps of contacting a surface to be decontaminated with a solution of hydrogen peroxide and water; and subsequently wiping the solution from the surface. Preferably, the solution consists essentially of hydrogen peroxide and water. The solution may also be sprayed on a surface, or applied first to a paper towel or the like, and then applied to a surface. The concentration of the hydrogen peroxide solution is dilute. Concentrations of about 0.5% to about 30% are preferred. A concentration of about 3% is most highly preferred.

### BACKGROUND

This Nonprovisional application claims priority under 35 U.S.C. § 119(e) on U.S. Provisional Application No(s). 60/724,302 filed on Oct. 7, 2005, the entire contents of which are hereby incorporated by reference.

Decontamination of nucleic acids from surfaces involved in the Polymerase Chain Reaction (PCR) technique, and other related DNA amplification techniques, is extremely important. PCR, and related techniques, may amplify extraneous nucleic acids that, for example, have been carried over from a previous amplification. This can lead to false positive results and mistyping. In prior methods of surface decontamination, expensive, difficult to handle solutions have generally been deployed as the decontamination agent. In most cases, post-decontamination steps involving cleaning decontamination reagent residue are also required. There is a need in the industry for a decontamination process that is inexpensive, easy to use, and that utilizes readily available, user-friendly reagents.

### DESCRIPTION OF THE INVENTION

The present invention involves treating a surface with dilute aqueous hydrogen peroxide solution to remove nucleic acid contamination from the surface area. Dilute solutions of hydrogen peroxide are inexpensive, easy to handle, and are extremely effective at removing nucleic acid contamination from a surface. Preferred solutions of this invention consist essentially of hydrogen peroxide and water. These solutions may be used without the need of additional surface cleaning steps that are generally necessary with other decontamination solutions. The solutions of the present invention do not leave a residue that can interfere with future amplifications.

Accordingly, the present invention provides a method of reducing nucleic acid contamination on a surface, which comprises the steps of contacting a surface to be decontaminated with a solution of hydrogen peroxide and water; and subsequently wiping the solution from the surface. Preferably, the solution consists essentially of hydrogen peroxide and water. The solution may also be sprayed on a surface (e.g., a vertical surface), or applied first to a paper towel or the like, and then applied to a surface, and then wiped off.

In a typical embodiment the hydrogen peroxide solution is allowed to dry for at least about three minutes before wiping. However, the hydrogen peroxide solution may be allowed to dry for periods of 30 minutes, or an hour, or more.

The concentration of the hydrogen peroxide solution is dilute. Concentrations of about 0.5% to about 30% are preferred. More preferred concentrations are in the approximately 2% to approximately 10% range. A concentration of about 3% is most highly preferred.

Hydrogen Peroxide

The hydrogen peroxide solutions of the present invention may be readily, and inexpensively, obtained from commercial sources. For example, Hydrogen Peroxide 3% available from VWR, West Chester, Pa. (Cat. No. VW4540-2) may be used. However, the use of any commercial, generally available, solution of aqueous hydrogen peroxide is contemplated by the present invention. The hydrogen peroxide solution may also contain additional additives (stabilizers, etc.) that are commonly used in commercial hydrogen peroxide solutions.

A solution “consisting essentially of” hydrogen peroxide and water may contain further components that are unrelated to the invention, such as, for example, a stabilizer to prevent degradation of the hydrogen peroxide, but will not contain further oxidants such as bleach or surfactants or enzymes.

Surfaces

The surfaces and items that can be treated are any that are typically found in a laboratory environment. Preferably this includes any surface that would be present in the practice of nucleic acid amplification. This would include metal, glass, plastic, and ceramic surfaces. This would preferably include surfaces on laboratory benches, instruments, and equipment. This would also include surfaces in pipettors (including automated pipettors) used in nucleic acid amplification. For example, an instrument such as the BD ProbeTec™ ET Pipettor manufactured by Becton, Dickinson and Company, and the like, are in view. This would also include surfaces in arrays, microarrays, and microwells that are used in nucleic acid amplification.

Contaminants

The contaminants that can be cleaned by the present methods include any nucleic acid based contaminant. This would preferably include residual contaminants that may be present on the surfaces of laboratory equipment related to DNA amplification experiments. This especially includes any residual contamination that can interfere with a subsequent enzymatic reaction. The present methods can also decontaminate surfaces and items that are contaminated with radioactive contaminants.

Preferred Embodiments

In one preferred embodiment, the hydrogen peroxide solution is contacted with the surface to be decontaminated, the solution is allowed to dry, and is then wiped away in a final step. In this embodiment further wiping or cleaning of the surface is not performed. Thus, the present invention eliminates the need for further cleaning steps that are often required to remove residue left by the decontamination solution itself.

In another preferred embodiment, the hydrogen peroxide solution is thoroughly contacted with the surface to be decontaminated and then the surface is rinsed with water before wiping and drying.

In yet another preferred embodiment, an item to be decontaminated is soaked in the hydrogen peroxide solution and then is drained, rinsed with water, and dried.

In yet another preferred embodiment, a wipe or towel is first soaked in the hydrogen peroxide solution and then the surface to be cleaned is wiped with the wipe or towel. If needed or desirable for a particular application, the surface can then be further wiped with a dry towel or wipe, or with a towel soaked with water, or the surface can be rinsed with water.

### EXAMPLES

### General Materials and Methods

In a typical test procedure, a surface area containing 48—1″×1″ squares is contaminated using 10×10³ copies/mL GC plasmid. The plasmid stock is diluted to a 10×10² copies/mL concentration and applied to the surface. Two swabs from each square are taken to ensure the surface is contaminated. Dilute hydrogen peroxide is then applied to the contaminated surface area and used as a decontamination reagent. An additional two swabs are taken from each square and then tested on the BD ProbeTec™ ET. In one test, 93/96 swabs tested negative in both assays demonstrating a reduction rate of 97%.

All monoplex runs incorporated Amplification Control (AC) microwells to ensure that the hydrogen peroxide was not interfering with the Strand Displacement Amplification reaction. There were no AC indeterminates throughout the study. Therefore, it can be concluded that hydrogen peroxide does not cause inhibition in the assay and should not be considered a risk factor to the product.

Definitions

GC: Neisseria gonorrhoeae. AC: Amplification Control. IAC: Internal Amplification Control. Monoplex: GC assay with an external amplification control (AC). Diplex (Qx): GC assay with an internal amplification control (IAC). MOTA: Method Other Than Acceleration. PAT: Passes After Threshold. H₂O₂: Hydrogen Peroxide. SD: Sample Diluent (potassium phosphate buffer) w/DMSO (10%) (CT/GC kit component).

Materials

The BD ProbeTec™ ET System is a robotic, high throughput, real-time nucleic acid amplification system, manufactured by Becton, Dickinson and Company, Franklin Lakes, N.J. A set of accessories for the system is available from the manufacturer for the detection of Chlamydia trachomatis (CT) and Neisseria gonorrhoeae (GC) in clinical specimens. The BD Viper™ Sample Processor is a robotic system that automates the sample handling associated with high volume amplified molecular testing and is also available from Becton Dickinson. The BD Viper™ Sample Processor can be used with the BD ProbeTec™ ET system for detection of Chlamydia trachomatis and Neisseria gonorrhoeae. BD ProbeTec™ ET GC Priming and Amplification Microwells, BD ProbeTec™ ET AC Priming and Amplification Microwells, BD ProbeTec™ ET GCQx Priming and Amplification Microwells, BD ProbeTec™ ET CT/GC Positive Control, BD ProbeTec™ ET CTQx/GCQx Positive Control, BD ProbeTec™ ET Negative Control, BD ProbeTec™ ET Sample Diluent Tubes, BD ProbeTec™ ET Pipette Tips, BD ProbeTec™ ET Chlamydia trachomatis and Neisseria gonorrhoeae (CT/GC) are accessories that are useful with the BD ProbeTec™ and BD Viper™ systems. The above products are further described and can be ordered through the Becton Dickinson web site.

Amplified DNA Assay Endocervical Specimen Collection and Dry Transport Kit Equipment

BD ProbeTec™ ET Instrument System. BD ProbeTec™ ET Matrix Pipettor.

Additional materials:

VWR™ Hydrogen Peroxide 3%, Stabilized (Cat. No. VW3540-2) GC Plasmid Stock—10×10³ copies/μL concentration Nuclease-free Water LS Pipette Tips (100 μL-1000 μL).

General Testing Procedure

Use thin labeling tape to set-up a “grid” containing six 1″×1″ by eight 1″×1″ squares creating a total of 48—1″×1″ squares. Dilute a 10×10³ GC plasmid stock to a 10×10² copies/mL concentration. Dispense 100 mL of the dilute GC plasmid stock onto each square of the grid. Use the cleaning swab provided in the BD ProbeTec™ ET Chlamydia trachomatis and Neisseria gonorrhoeae (CT/GC) Amplified DNA Assay Endocervical Specimen Collection and Dry Transport Kit to spread the 100 mL of GC plasmid evenly across each individual square. Allow the grid surface to dry completely. (30 min-1 hr.) Using the endocervical swab provided in the BD ProbeTec™ ET Chlamydia trachomatis and Neisseria gonorrhoeae (CT/GC) Amplified DNA Assay Endocervical Specimen Collection and Dry Transport Kit take two swab samples from each square, totaling 96 samples. Express each swab into a BD ProbeTec™ ET CT/GC Swab Diluent Tube. Cap and vortex each tube for 5 seconds. Prepare a negative and positive control tube by adding 2 mL of BD ProbeTec™ CT/GC Sample Diluent to each tube and vortexing for 5 seconds. Heat lyse all samples and controls at 114° C. for 30 minutes. Then allow samples to cool at room temperature for 15 minutes-6 hours. Unscrew sample diluent caps and discard. Set up priming and amplification plates in accordance to Appendix I. Perform the amplification reactions. Each swab must test positive in at least one of the two assays (GC monoplex/GC Qx) to be considered positive.

Break seal and liberally apply dilute (3%) Hydrogen Peroxide over entire grid surface and allow to stand for three minutes. Wipe surface with Teri-Towel in a one-directional motion. Repeat General Testing Procedure described above.

### Example 1

### Comparison of Eliminase™ vs. Hydrogen Peroxide

A total of 6 1′×2′ blocks were measured and taped off on a laboratory counter top. Each cleaning method is represented by 3 blocks as shown below:

| | | |
|---|---|---|
| 1 | 3 | 5 |
| 2 | 4 | 6 |

1: Hydrogen Peroxide
2: Eliminase ™
3: Eliminase ™
4: Hydrogen Peroxide
5: Hydrogen Peroxide
6: Eliminase ™

48 Positive Controls were resuspended with 1 mL CT/GC SD, lysed for 30 min. at 114° C., then cooled for 15 min. 8 (8 mL) Positive Controls were “spilled” on to each of the 6 blocks and spread using a swab. The blocks were allowed to dry for approx. 30 minutes.

Before cleaning, 8 swab samples were taken from each block and expressed into a pre-filled SD tube. The blocks were then cleaned according to the designated cleaning method for each block. After cleaning, an additional 8 swab samples were taken from each block and expressed into a pre-filled SD tube. 2 mL of SD was added to CT/GC Positive and Negative Controls. The tubes were then lysed in lysing block at 114° C. for 30 minutes and cooled for 15 minutes. 150 uL sample was added to the priming wells to be tested, and then incubated at room temp for 20 min. 150 uL of positive and negative CT/GC controls were added to control priming wells.

The priming plates were placed on a 72° C. heat block and amp plates were placed on a 54° C. heat block for 10 min. 100 uL of priming mix was transferred to corresponding amplification wells. The plates were sealed and run in the ProbeTec instrument for 60 minutes. Both Eliminase and hydrogen peroxide were effective decontamination reagents. Both reduced the number of contaminated swabs by 100%. The results are shown in TABLES 1 and 2 in the RESULTS section.

### Example 2

### The Effectiveness of Dilute Hydrogen Peroxide as a Decontamination Reagent

A total of 48 1″×1″ blocks were measured and taped off on the counter top. A GC plasmid stock with a concentration of 10.11×10³ was used to make the GC plasmid dilution. A 1:10 dilution was used to create a final GC plasmid concentration of 10.11×10². To create enough volume to “contaminate” the 48 blocks, 500 uL of the GC plasmid stock and 4500 uL of deionized water were combined.

The blocks were allowed to dry for approx. 1 hour. Two swab samples were taken from each block and expressed into a pre-filled SD tube. 2 mL of SD was added to CT/GC Positive and Negative Controls and Qx Positive Control. The tubes were then lysed in the lysing block at 114° C. for 30 minutes and cooled for 15 minutes. 150 uL sample was added to the priming wells to be tested, and then incubated at room temperature for 20 min.

150 uL of positive and negative CT/GC controls were added to control priming wells. The priming plates were placed on a 72° C. heat block and amplification plates were placed on a 54° C. heat block for 10 min. 100 uL of priming mix was transferred to corresponding amplification wells. The plates were sealed and run in ProbeTec® instrument for 60 minutes. Both GC monoplex and GC diplex were tested in this study. Dilute hydrogen peroxide was liberally poured onto the 48 contaminated squares, allowed to stand for 3 minutes, then wiped away in a one-directional motion. These steps were repeated and then 2 swabs were taken from each square, processed and tested. 93/96 swabs tested negative in both assays after cleaning the surface with hydrogen peroxide resulting in a reduction rate of 97%. TABLES 3-10 in the RESULTS section show data obtained from before cleaning. TABLES 11-18 show data from after cleaning.

### Example 3

### Potency Stability of Hydrogen Peroxide

The potency stability of the hydrogen peroxide has also been verified. The same method used as described above in Example 2 was used on Days 1, 3, 5, and 8. On day one, the seal on the hydrogen peroxide bottle was broken and the same bottle was used for the duration of the testing. The positivity reduction rates for each day were as follows: Day 1—95%, Day 3—81%, Day 5—88%, and Day 8—81%. Although, there appears to be a slight decline in the potency of the hydrogen peroxide in reducing the contamination, the results show that the effectiveness of the solution remains very high over this length of time. The results are shown in Tables 19-82 in the RESULTS section.

### Results

TABLE 1
Eliminase

| | 2 | | 2 | | 3 | | 3 | | 6 | | 6 | |
|---|---|---|---|---|---|---|---|---|---|---|---|---|
| | Before | | After | | Before | | After | | Before | | After | |
| Swab/Sample # | CT | GC | CT | GC | CT | GC | CT | GC | CT | GC | CT | GC |
| 1 | 20770 | 20282 | 244 | 165 | 36413 | 16583 | 313 | 540 | 31595 | 2521 | 531 | 367 |
| 2 | 3172 | 450 | 95 | 275 | 24613 | 14351 | 105 | 540 | 28250 | 11942 | 711 | 555 |
| 3 | 31679 | 246 | 25 | 250 | 4895 | 525 | 250 | 195 | 27549 | 123964 | 261 | 307 |
| 4 | 14981 | 4378 | 13 | 95 | 19757 | 7859 | 14 | 100 | 8157 | 17550 | 204 | 717 |
| 5 | 30782 | 672 | 40 | 98 | 2822 | 6755 | 271 | 140 | 30287 | 441 | 530 | 708 |
| 6 | 32988 | 4184 | 106 | 14 | 15368 | 13670 | 357 | 252 | 35619 | 370 | 15 | 412 |
| 7 | 30822 | 14154 | 416 | 253 | 33080 | 240 | 450 | 152 | 26073 | 765 | 467 | 493 |
| 8 | 15780 | 1978 | 270 | 387 | 32095 | 7501 | 272 | 301 | 5550 | 491 | 438 | 507 |

Total # of Positives (MOTA over 2,000)

| | | |
|---|---|---|
| Before cleaning: | After cleaning: | Reduction percentage: |

| | | | | |
|---|---|---|---|---|
| CT | 24 | CT | 0 | 100% |
| GC | 14 | GC | 0 | 100% |

TABLE 2
Hydrogen Peroxide

| | 1 | | 1 | | 4 | | 4 | | 5 | | 5 | |
|---|---|---|---|---|---|---|---|---|---|---|---|---|
| | Before | | After | | Before | | After | | Before | | After | |
| Swab/Sample # | CT | GC | CT | GC | CT | GC | CT | GC | CT | GC | CT | GC |
| 1 | 29212 | 3585 | 124 | 333 | 17100 | 722 | 564 | 734 | 10 | 8269 | 713 | 258 |
| 2 | 30976 | 202 | 325 | 467 | 26314 | 414 | 268 | 797 | 2832 | 23837 | 4115 | 728 |
| 3 | 26248 | 22051 | 317 | 408 | 7228 | 20329 | 632 | 882 | 5982 | 25144 | 376 | 244 |
| 4 | 4390 | 3531 | 370 | 286 | 5516 | 14712 | 373 | 523 | 14536 | 24993 | 651 | 296 |
| 5 | 241 | 221 | 564 | 407 | 4908 | 13976 | 224 | 466 | 25 | 19496 | 763 | 509 |
| 6 | 29537 | 593 | 289 | 455 | 7354 | 490 | 1112 | 593 | 911 | 12677 | 556 | 254 |
| 7 | 32499 | 5018 | 111 | 491 | 8974 | 16556 | 502 | 342 | 2858 | 12808 | 312 | 525 |
| 8 | 30944 | 592 | 436 | 597 | 21606 | 4190 | 847 | 399 | 3961 | 580 | 770 | 395 |

Total # of Positives (MOTA over 2,000)

| | | |
|---|---|---|
| Before cleaning: | After cleaning: | Reduction percentage: |

| | | | | |
|---|---|---|---|---|
| CT | 20 | CT | 0 | 100% |
| GC | 16 | GC | 0 | 100% |

Conclusions: Both Eliminase and hydrogen peroxide were effective decontamination reagents. Both reduced the number of contaminated swabs by 100%.

TABLE 3
Monoplex

| Swab # | GC | AC |
|---|---|---|
| 1 | 31364 | 50394 |
| 2 | 35493 | 42726 |
| 3 | 33960 | 48992 |
| 4 | 32278 | 43363 |
| 5 | 33599 | 36893 |
| 6 | 25119 | 38112 |
| 7 | 23628 | 43983 |
| 8 | 24130 | 43865 |
| 9 | 33329 | 39304 |
| 10 | 27083 | 46092 |
| 11 | 31278 | 44114 |
| 12 | 33784 | 34758 |
| 13 | 29863 | 36999 |
| 14 | 31960 | 30135 |
| 15 | 28277 | 25647 |
| 16 | 21782 | 30744 |
| 17 | 44078 | 38812 |
| 18 | 41871 | 38268 |
| 19 | 39770 | 25318 |
| 20 | 34484 | 33183 |
| 21 | 39340 | 39543 |
| 22 | 41163 | 31320 |
| 23 | 27470 | 37782 |
| 24 | 32076 | 30784 |

TABLE 4
Diplex

| Swab # | dGC | IAC |
|---|---|---|
| 1 | 0 | 48.3 |
| 2 | 0 | 47.3 |
| 3 | 0 | 47.4 |
| 4 | 0 | 47.7 |
| 5 | 39.4 | 44.8 |
| 6 | 31.6 | 45.3 |
| 7 | 35.1 | 44.2 |
| 8 | 25.6 | 45.3 |
| 9 | 25 | 45.3 |
| 10 | 16 | 46.4 |
| 11 | 28.9 | 44.8 |
| 12 | 0 | 46.5 |
| 13 | 31.8 | 44.3 |
| 14 | 38.5 | 39.9 |
| 15 | 26.7 | 45 |
| 16 | 33.5 | 43 |
| 17 | 30.1 | 45.2 |
| 18 | 28.8 | 44.6 |
| 19 | 27.8 | 45.4 |
| 20 | 22.1 | 44.8 |
| 21 | 23.9 | 45 |
| 22 | 20.7 | 44.2 |
| 23 | 26.9 | 44 |
| 24 | 29.3 | 42.7 |

TABLE 5
Monoplex

| Swab # | GC | AC |
|---|---|---|
| 25 | 33856 | 33496 |
| 26 | 27569 | 37453 |
| 27 | 31136 | 34121 |
| 28 | 15777 | 39507 |
| 29 | 34828 | 24126 |
| 30 | 31661 | 28196 |
| 31 | 24737 | 30375 |
| 32 | 19459 | 26424 |
| 33 | 22536 | 34749 |
| 34 | 32804 | 42086 |
| 35 | 27411 | 40577 |
| 36 | 42416 | 39354 |
| 37 | 35807 | 37442 |
| 38 | 29762 | 34159 |
| 39 | 25496 | 30281 |
| 40 | 20446 | 31083 |
| 41 | 30029 | 36878 |
| 42 | 15555 | 27768 |
| 43 | 26864 | 32447 |
| 44 | 21429 | 42172 |
| 45 | 24216 | 33836 |
| 46 | 25102 | 35481 |
| 47 | 24764 | 27891 |
| 48 | 26986 | 29736 |

TABLE 6
Diplex

| Swab # | dGC | IAC |
|---|---|---|
| 25 | 38.1 | 40.1 |
| 26 | 32.7 | 43 |
| 27 | 27.7 | 44.3 |
| 28 | 30.5 | 43.4 |
| 29 | 33 | 43.3 |
| 30 | 33 | 42 |
| 31 | 39 | 41.9 |
| 32 | 30 | 43.9 |
| 33 | 33.9 | 43.6 |
| 34 | 36.1 | 42.9 |
| 35 | 32.8 | 44.3 |
| 36 | 37.1 | 44.9 |
| 37 | 25.2 | 43.9 |
| 38 | 36.9 | 42.2 |
| 39 | 34 | 41.5 |
| 40 | 8 | 43 |
| 41 | 29.8 | 44.6 |
| 42 | 39.3 | 39.6 |
| 43 | 18.6 | 43.1 |
| 44 | 19.4 | 43.7 |
| 45 | 18.1 | 42.4 |
| 46 | 26.7 | 42.9 |
| 47 | 31.6 | 42 |
| 48 | 25 | 42.4 |

TABLE 7
Monoplex

| Swab # | GC | AC |
|---|---|---|
| 49 | 12784 | 38202 |
| 50 | 24748 | 43408 |
| 51 | 25867 | 31233 |
| 52 | 32685 | 36069 |
| 53 | 34427 | 40106 |
| 54 | 31736 | 31169 |
| 55 | 36900 | 34232 |
| 56 | 26713 | 30842 |
| 57 | 30474 | 36143 |
| 58 | 45265 | 36869 |
| 59 | 30729 | 29538 |
| 60 | 36792 | 35887 |
| 61 | 35771 | 34762 |
| 62 | 36325 | 43327 |
| 63 | 35065 | 31751 |
| 64 | 33028 | 34615 |
| 65 | 35178 | 36599 |
| 66 | 35010 | 37790 |
| 67 | 35754 | 47546 |
| 68 | 33550 | 37351 |
| 69 | 35627 | 32382 |
| 70 | 36684 | 42521 |
| 71 | 44428 | 34987 |
| 72 | 31486 | 35386 |

TABLE 8
Diplex

| Swab # | dGC | IAC |
|---|---|---|
| 49 | 32.4 | 40.7 |
| 50 | 32.9 | 42.6 |
| 51 | 30 | 41.8 |
| 52 | 33.6 | 42 |
| 53 | 28.2 | 43.5 |
| 54 | 28.1 | 41 |
| 55 | 31.3 | 43.2 |
| 56 | 35.3 | 41.1 |
| 57 | 37.6 | 43.3 |
| 58 | 12.7 | 43.9 |
| 59 | 30.3 | 43.1 |
| 60 | 28.5 | 41 |
| 61 | 25.2 | 40.5 |
| 62 | 32.1 | 39.5 |
| 63 | 32.6 | 36.7 |
| 64 | 29.5 | 36.6 |
| 65 | 27.2 | 44.1 |
| 66 | 30 | 44.5 |
| 67 | 33.7 | 41.8 |
| 68 | 34.8 | 42.4 |
| 69 | 29.9 | 42.4 |
| 70 | 16.7 | 43.3 |
| 71 | 6 | 42.9 |
| 72 | 38.2 | 41.7 |

TABLE 9
Monoplex

| Swab # | GC | AC |
|---|---|---|
| 73 | 34515 | 33506 |
| 74 | 39151 | 42188 |
| 75 | 35729 | 45742 |
| 76 | 42110 | 33697 |
| 77 | 43137 | 35236 |
| 78 | 38550 | 29939 |
| 79 | 34194 | 34515 |
| 80 | 28716 | 30208 |
| 81 | 31626 | 37949 |
| 82 | 20845 | 32903 |
| 83 | 30901 | 31340 |
| 84 | 30293 | 25968 |
| 85 | 26748 | 19044 |
| 86 | 24083 | 14329 |
| 87 | 22452 | 16205 |
| 88 | 20362 | 13921 |
| 89 | 31049 | 32319 |
| 90 | 25964 | 36240 |
| 91 | 28274 | 39771 |
| 92 | 28831 | 38660 |
| 93 | 26130 | 27489 |
| 94 | 21889 | 29938 |
| 95 | 15220 | 29318 |
| 96 | 18217 | 25401 |

TABLE 10
Diplex

| Swab # | dGC | IAC |
|---|---|---|
| 73 | 32.7 | 44.2 |
| 74 | 26.7 | 43.5 |
| 75 | 31.6 | 41.6 |
| 76 | 28.9 | 42.8 |
| 77 | 33.8 | 40.9 |
| 78 | 34.1 | 38.9 |
| 79 | 31.3 | 40.5 |
| 80 | 0 | 41.4 |
| 81 | 31.7 | 44.9 |
| 82 | 29.7 | 44.4 |
| 83 | 33.3 | 42.5 |
| 84 | 36.6 | 42.9 |
| 85 | 31.5 | 43.1 |
| 86 | 29.8 | 43 |
| 87 | 31.8 | 42 |
| 88 | 33.2 | 38 |
| 89 | 38 | 43.1 |
| 90 | 28.8 | 42.8 |
| 91 | 35.4 | 43 |
| 92 | 24.9 | 43.2 |
| 93 | 36.4 | 41.5 |
| 94 | 33.8 | 41.6 |
| 95 | 33.9 | 40.3 |
| 96 | 26.5 | 39.9 |

TABLE 11
Monoplex

| Swab # | GC | AC |
|---|---|---|
| 1 | 283 | 31104 |
| 2 | 102 | 35019 |
| 3 | 281 | 26828 |
| 4 | 151 | 21997 |
| 5 | 593 | 22532 |
| 6 | 536 | 21070 |
| 7 | 319 | 20845 |
| 8 | 1256 | 19485 |
| 9 | 340 | 31008 |
| 10 | 118 | 36024 |
| 11 | 207 | 39055 |
| 12 | 245 | 36666 |
| 13 | 31 | 50100 |
| 14 | 120 | 33414 |
| 15 | 316 | 42091 |
| 16 | 220 | 30603 |
| 17 | 391 | 44918 |
| 18 | 120 | 43951 |
| 19 | 431 | 45014 |
| 20 | 179 | 44951 |
| 21 | 406 | 43860 |
| 22 | 563 | 37072 |
| 23 | 568 | 34083 |
| 24 | 16840 | 43604 |

TABLE 12
Diplex

| Swab # | dGC | IAC |
|---|---|---|
| 1 | 0 | 34 |
| 2 | 0 | 40.3 |
| 3 | 0 | 39 |
| 4 | 0 | 40.4 |
| 5 | 0 | 40.4 |
| 6 | 0 | 42.1 |
| 7 | 0 | 39.4 |
| 8 | 0 | 39.5 |
| 9 | 0 | 42.1 |
| 10 | 0 | 40.6 |
| 11 | 0 | 40.8 |
| 12 | 0 | 39.9 |
| 13 | 0 | 40.2 |
| 14 | 0 | 42.1 |
| 15 | 0 | 38.2 |
| 16 | 0 | 37.6 |
| 17 | 0 | 36.4 |
| 18 | 0 | 40.1 |
| 19 | 0 | 39.5 |
| 20 | 0 | 40.8 |
| 21 | 0 | 40.1 |
| 22 | 0 | 36.7 |
| 23 | 0 | 40.7 |
| 24 | 0 | 37 |

TABLE 13
Monoplex

| Swab # | GC | AC |
|---|---|---|
| 25 | 470 | 37526 |
| 26 | 274 | 39090 |
| 27 | 283 | 43810 |
| 28 | 410 | 38927 |
| 29 | 585 | 38668 |
| 30 | 107 | 38429 |
| 31 | 706 | 25856 |
| 32 | 212 | 37936 |
| 33 | 302 | 44990 |
| 34 | 463 | 42995 |
| 35 | 315 | 49850 |
| 36 | 324 | 36601 |
| 37 | 218 | 39498 |
| 38 | 552 | 34683 |
| 39 | 488 | 35960 |
| 40 | 531 | 30500 |
| 41 | 554 | 27198 |
| 42 | 638 | 31739 |
| 43 | 224 | 36759 |
| 44 | 455 | 34113 |
| 45 | 260 | 29577 |
| 46 | 181 | 35186 |
| 47 | 964 | 35460 |
| 48 | 3066 | 38114 |

TABLE 14
Diplex

| Swab # | dGC | IAC |
|---|---|---|
| 25 | 0 | 44.4 |
| 26 | 0 | 42.6 |
| 27 | 0 | 42.5 |
| 28 | 0 | 40.5 |
| 29 | 0 | 42.6 |
| 30 | 0 | 42 |
| 31 | 0 | 44.3 |
| 32 | 0 | 41.6 |
| 33 | 0 | 41.3 |
| 34 | 0 | 43.1 |
| 35 | 0 | 42.6 |
| 36 | 0 | 41.8 |
| 37 | 0 | 41.1 |
| 38 | 0 | 39.7 |
| 39 | 0 | 35.2 |
| 40 | 0 | 37.9 |
| 41 | 0 | 40.4 |
| 42 | 0 | 42.7 |
| 43 | 0 | 38.3 |
| 44 | 0 | 41.7 |
| 45 | 0 | 42.5 |
| 46 | 0 | 37.9 |
| 47 | 0 | 39.9 |
| 48 | 0 | 40.6 |

TABLE 15
Monoplex

| Swab # | GC | AC |
|---|---|---|
| 49 | 429 | 24785 |
| 50 | 351 | 32287 |
| 51 | 252 | 33821 |
| 52 | 77 | 38974 |
| 53 | 431 | 35841 |
| 54 | 242 | 33858 |
| 55 | 325 | 43736 |
| 56 | 187 | 35096 |
| 57 | 453 | 39494 |
| 58 | 192 | 36295 |
| 59 | 16605 | 40458 |
| 60 | 1 | 31242 |
| 61 | 17 | 31348 |
| 62 | 250 | 38853 |
| 63 | 52 | 35122 |
| 64 | 222 | 33951 |
| 65 | 551 | 31394 |
| 66 | 871 | 43460 |
| 67 | 124 | 42415 |
| 68 | 521 | 34260 |
| 69 | 722 | 37371 |
| 70 | 277 | 35209 |
| 71 | 188 | 41689 |
| 72 | 374 | 41145 |

TABLE 16
Diplex

| Swab # | dGC | IAC |
|---|---|---|
| 49 | 0 | 44.1 |
| 50 | 0 | 43.8 |
| 51 | 0 | 43.5 |
| 52 | 0 | 44.4 |
| 53 | 0 | 44 |
| 54 | 0 | 42.2 |
| 55 | 0 | 44.5 |
| 56 | 0 | 45 |
| 57 | 0 | 42.3 |
| 58 | 0 | 42.8 |
| 59 | 0 | 43.6 |
| 60 | 0 | 43.1 |
| 61 | 0 | 40.8 |
| 62 | 0 | 41.3 |
| 63 | 0 | 41.8 |
| 64 | 0 | 39.2 |
| 65 | 0 | 41.8 |
| 66 | 0 | 42.3 |
| 67 | 0 | 44.1 |
| 68 | 0 | 43.2 |
| 69 | 0 | 43.3 |
| 70 | 0 | 43.7 |
| 71 | 0 | 41.9 |
| 72 | 0 | 38 |

TABLE 17
Monoplex

| Swab # | GC | AC |
|---|---|---|
| 73 | 478 | 41807 |
| 74 | 744 | 40794 |
| 75 | 614 | 38845 |
| 76 | 168 | 36003 |
| 77 | 373 | 32306 |
| 78 | 461 | 31253 |
| 79 | 329 | 35115 |
| 80 | 425 | 32632 |
| 81 | 582 | 44919 |
| 82 | 641 | 44467 |
| 83 | 552 | 42058 |
| 84 | 42 | 42134 |
| 85 | 405 | 35646 |
| 86 | 308 | 39475 |
| 87 | 361 | 33523 |
| 88 | 312 | 23408 |
| 89 | 288 | 49240 |
| 90 | 52 | 40311 |
| 91 | 68 | 37115 |
| 92 | 191 | 35237 |
| 93 | 83 | 38290 |
| 94 | 2 | 39829 |
| 95 | 1 | 39750 |
| 96 | 60 | 51462 |

TABLE 18
Diplex

| Swab # | dGC | IAC |
|---|---|---|
| 73 | 0 | 40 |
| 74 | 0 | 39.4 |
| 75 | 0 | 44.5 |
| 76 | 0 | 44.6 |
| 77 | 0 | 43 |
| 78 | 0 | 41.9 |
| 79 | 0 | 43.7 |
| 80 | 0 | 35.4 |
| 81 | 0 | 39.9 |
| 82 | 0 | 38.7 |
| 83 | 0 | 40.4 |
| 84 | 0 | 42 |
| 85 | 0 | 42.3 |
| 86 | 0 | 42 |
| 87 | 0 | 38.4 |
| 88 | 0 | 39.6 |
| 89 | 0 | 39.8 |
| 90 | 0 | 37.2 |
| 91 | 0 | 41.9 |
| 92 | 0 | 42.6 |
| 93 | 0 | 40 |
| 94 | 0 | 40 |
| 95 | 0 | 42.9 |
| 96 | 0 | 39.7 |

### Example 3

### Day 1— Before Cleaning

All swabs are positive for GC in at least one assay (either monoplex or diplex)

TABLE 19
Monoplex

| Swab # | GC | AC |
|---|---|---|
| 1 | 29422 | 23955 |
| 2 | 34169 | 34733 |
| 3 | 34500 | 34463 |
| 4 | 60421 | 32173 |
| 5 | 36864 | 35684 |
| 6 | 37397 | 32498 |
| 7 | 42297 | 34432 |
| 8 | 41069 | 36655 |
| 9 | 25526 | 22095 |
| 10 | 32223 | 23860 |
| 11 | 33152 | 29639 |
| 12 | 32868 | 26648 |
| 13 | 31303 | 39657 |
| 14 | 33661 | 28446 |
| 15 | 35865 | 26365 |
| 16 | 32361 | 34905 |
| 17 | 23508 | 42571 |
| 18 | 27570 | 45438 |
| 19 | 29546 | 37838 |
| 20 | 28973 | 43077 |
| 21 | 38210 | 40570 |
| 22 | 27177 | 2777B |
| 23 | 30073 | 33056 |
| 24 | 29102 | 32163 |

TABLE 20
Diplex

| Swab # | dGC | IAC |
|---|---|---|
| 1 | 36.1 | 40.4 |
| 2 | 38.5 | 39.6 |
| 3 | 39.4 | 38.7 |
| 4 | 38 | 39.1 |
| 5 | 37.7 | 36.4 |
| 6 | 36.2 | 39.4 |
| 7 | 39 | 37.3 |
| 8 | 38.8 | 41.5 |
| 9 | 37.3 | 39.8 |
| 10 | 36.1 | 37.3 |
| 11 | 37.3 | 34.4 |
| 12 | 37.3 | 35.6 |
| 13 | 34.8 | 38.9 |
| 14 | 35.5 | 38.9 |
| 15 | 38.7 | 33.4 |
| 16 | 32.6 | 41.9 |
| 17 | 35.9 | 38.1 |
| 18 | 34.7 | 40.2 |
| 19 | 38.4 | 32.5 |
| 20 | 36.7 | 39.8 |
| 21 | 37.3 | 36.6 |
| 22 | 34.4 | 38.5 |
| 23 | 35.7 | 39.2 |
| 24 | 39.6 | 32.7 |

TABLE 21
Monoplex

| Swab # | GC | AC |
|---|---|---|
| 25 | 35894 | 46602 |
| 26 | 38841 | 50406 |
| 27 | 39232 | 51088 |
| 28 | 34687 | 47266 |
| 29 | 39193 | 52651 |
| 30 | 31448 | 38943 |
| 31 | 46369 | 51620 |
| 32 | 42591 | 54348 |
| 33 | 27952 | 37645 |
| 34 | 29599 | 47150 |
| 35 | 33911 | 49571 |
| 36 | 29796 | 35096 |
| 37 | 31922 | 40339 |
| 38 | 25925 | 31743 |
| 39 | 25725 | 26851 |
| 40 | 28542 | 29505 |
| 41 | 32265 | 24964 |
| 42 | 33388 | 31862 |
| 43 | 34465 | 41020 |
| 44 | 36121 | 28816 |
| 45 | 45894 | 30027 |
| 46 | 46652 | 34509 |
| 47 | 36485 | 28747 |
| 48 | 34370 | 41159 |

TABLE 22
Diplex

| Swab # | dGC | IAC |
|---|---|---|
| 25 | 38.6 | 36.6 |
| 26 | 33.4 | 35.4 |
| 27 | 37.9 | 40.2 |
| 28 | 37 | 35.4 |
| 29 | 35.7 | 34.6 |
| 30 | 38.3 | 41.5 |
| 31 | 37.4 | 39.3 |
| 32 | 33.8 | 41.5 |
| 33 | 36.8 | 40.4 |
| 34 | 33.4 | 38.8 |
| 35 | 37.9 | 36 |
| 36 | 37 | 39.6 |
| 37 | 35.7 | 41.8 |
| 38 | 38.3 | 36.7 |
| 39 | 3.4 | 37.9 |
| 40 | 33.8 | 39.8 |
| 41 | 36.6 | 40.1 |
| 42 | 37.4 | 37.3 |
| 43 | 35.3 | 38.6 |
| 44 | 34.6 | 41.1 |
| 45 | 38.7 | 39.4 |
| 46 | 37 | 36.9 |
| 47 | 37.7 | 38.6 |
| 48 | 38 | 41.1 |

TABLE 23
Monoplex

| Swab # | GC | AC |
|---|---|---|
| 49 | 15975 | 26558 |
| 50 | 20202 | 32382 |
| 51 | 21074 | 26215 |
| 52 | 24646 | 29210 |
| 53 | 22727 | 32027 |
| 54 | 24899 | 27500 |
| 55 | 27316 | 35641 |
| 56 | 24139 | 24061 |
| 57 | 35224 | 27628 |
| 58 | 37746 | 41375 |
| 59 | 37248 | 35698 |
| 60 | 38765 | 52546 |
| 61 | 37227 | 52762 |
| 62 | 36175 | 48067 |
| 63 | 42462 | 47615 |
| 64 | 38529 | 47503 |
| 65 | 49228 | 36441 |
| 66 | 34716 | 38208 |
| 67 | 42590 | 35867 |
| 68 | 31124 | 35637 |
| 69 | 39130 | 35033 |
| 70 | 34766 | 30135 |
| 71 | 50648 | 30106 |
| 72 | 32734 | 27707 |

TABLE 24
Diplex

| Swab # | dGC | IAC |
|---|---|---|
| 49 | 38.6 | 36.8 |
| 50 | 37.9 | 38.9 |
| 51 | 36.7 | 38.5 |
| 52 | 31.6 | 35.5 |
| 53 | 37.5 | 37.8 |
| 54 | 38.7 | 34.2 |
| 55 | 39.9 | 35.5 |
| 56 | 33.1 | 40.1 |
| 57 | 39.3 | 33.8 |
| 58 | 36.8 | 40.9 |
| 59 | 39.6 | 41.1 |
| 60 | 38.5 | 37.5 |
| 61 | 38.5 | 40.7 |
| 62 | 39.2 | 34.7 |
| 63 | 38.5 | 41.1 |
| 64 | 39.9 | 35 |
| 65 | 35.6 | 42.3 |
| 66 | 38.9 | 39 |
| 67 | 28.9 | 39.5 |
| 68 | 35.9 | 36.6 |
| 69 | 33.2 | 38.2 |
| 70 | 35 | 38.5 |
| 71 | 37.6 | 37.8 |
| 72 | 39.6 | 34.7 |

TABLE 25
Monoplex

| Swab # | GC | AC |
|---|---|---|
| 73 | 32979 | 41309 |
| 74 | 36966 | 53985 |
| 75 | 34823 | 41193 |
| 76 | 37498 | 62391 |
| 77 | 37068 | 50708 |
| 78 | 33964 | 44997 |
| 79 | 50102 | 46267 |
| 80 | 35765 | 39053 |
| 81 | 26910 | 27768 |
| 82 | 31258 | 32934 |
| 83 | 29923 | 31891 |
| 84 | 31396 | 31515 |
| 85 | 32027 | 37284 |
| 86 | 33364 | 40736 |
| 87 | 34679 | 42714 |
| 88 | 35762 | 40487 |
| 89 | 38270 | 21807 |
| 90 | 33906 | 32713 |
| 91 | 43495 | 34213 |
| 92 | 46868 | 30389 |
| 93 | 30472 | 25301 |
| 94 | 46633 | 30108 |
| 95 | 53505 | 22971 |
| 96 | 47014 | 18459 |

TABLE 26
Diplex

| Swab # | dGC | IAC |
|---|---|---|
| 73 | 39.8 | 40.4 |
| 74 | 37.6 | 41.7 |
| 75 | 39.6 | 39.7 |
| 76 | 37.6 | 38.4 |
| 77 | 38.8 | 39.1 |
| 78 | 36.9 | 40.4 |
| 79 | 36.7 | 37.4 |
| 80 | 38.1 | 38.3 |
| 81 | 38.3 | 35.5 |
| 82 | 37.5 | 36.3 |
| 83 | 38.6 | 39.2 |
| 84 | 37 | 34.6 |
| 85 | 38.7 | 34.2 |
| 86 | 35 | 37 |
| 87 | 38.4 | 39 |
| 88 | 38.1 | 40.7 |
| 89 | 37.8 | 38 |
| 90 | 38.6 | 37.3 |
| 91 | 36.4 | 39.3 |
| 92 | 34.9 | 40.2 |
| 93 | 37 | 40.7 |
| 94 | 37.6 | 37.8 |
| 95 | 38.8 | 38.4 |
| 96 | 35.8 | 39.9 |

### Example 3

### Day 1— After Cleaning

Comments:

91/96 swabs tested in negative in both assays after cleaning the surface with hydrogen peroxide resulting in a reduction rate of 95%.

Dilute Hydrogen Peroxide was liberally poured onto the 48 squares, allowed to stand for 3 minutes, then wiped away in a one-directional motion. These steps were repeated and then 2 swabs were taken from each square, processed and tested.

TABLE 27
Monoplex

| Swab # | GC | AC |
|---|---|---|
| 1 | 1204 | 42294 |
| 2 | 490 | 40421 |
| 3 | 533 | 45944 |
| 4 | 152 | 46735 |
| 5 | 498 | 45032 |
| 6 | 246 | 35374 |
| 7 | 225 | 35381 |
| 8 | 582 | 37428 |
| 9 | 308 | 23294 |
| 10 | 299 | 26372 |
| 11 | 189 | 30289 |
| 12 | 1244 | 33642 |
| 13 | 321 | 31469 |
| 14 | 495 | 29552 |
| 15 | 460 | 26023 |
| 16 | 400 | 21632 |
| 17 | 220 | 53294 |
| 18 | 416 | 38017 |
| 19 | 317 | 47082 |
| 20 | 297 | 55486 |
| 21 | 484 | 42940 |
| 22 | 433 | 39194 |
| 23 | 310 | 42997 |
| 24 | 360 | 33393 |

TABLE 28
LE 30

| Swab # | dGC | IAC |
|---|---|---|
| 1 | 0 | 36.6 |
| 2 | 0 | 38.7 |
| 3 | 0 | 40.2 |
| 4 | 0 | 39.8 |
| 5 | 0 | 36.8 |
| 6 | 0 | 39.9 |
| 7 | 0 | 37 |
| 8 | 0 | 36.5 |
| 9 | 0 | 39.7 |
| 10 | 0 | 41.3 |
| 11 | 0 | 39.8 |
| 12 | 0 | 41.1 |
| 13 | 0 | 42.1 |
| 14 | 0 | 37.2 |
| 15 | 0 | 37.5 |
| 16 | 0 | 36.6 |
| 17 | 0 | 36.7 |
| 18 | 0 | 39.2 |
| 19 | 0 | 37.1 |
| 20 | 0 | 35.5 |
| 21 | 0 | 39.8 |
| 22 | 0 | 36.3 |
| 23 | 0 | 23.4 |
| 24 | 0 | 36.8 |

TABLE 29
Monoplex

| Swab # | GC | AC |
|---|---|---|
| 25 | 413 | 37998 |
| 26 | 205 | 43597 |
| 27 | 477 | 52248 |
| 28 | 241 | 45243 |
| 29 | 431 | 47610 |
| 30 | 294 | 51932 |
| 31 | 421 | 43744 |
| 32 | 554 | 48093 |
| 33 | 497 | 47045 |
| 34 | 237 | 58197 |
| 35 | 435 | 41834 |
| 36 | 516 | 47229 |
| 37 | 663 | 40510 |
| 38 | 433 | 55668 |
| 39 | 469 | 45576 |
| 40 | 791 | 48833 |
| 41 | 156 | 24085 |
| 42 | 324 | 22507 |
| 43 | 384 | 26965 |
| 44 | 363 | 32557 |
| 45 | 234 | 30607 |
| 46 | 522 | 29461 |
| 47 | 494 | 30017 |
| 48 | 149 | 25616 |

TABLE 30
Diplex

| Swab # | dGC | IAC |
|---|---|---|
| 25 | 0 | 40.3 |
| 26 | 0 | 39.8 |
| 27 | 0 | 39.2 |
| 28 | 0 | 39.5 |
| 29 | 0 | 41 |
| 30 | 0 | 37.4 |
| 31 | 0 | 37.5 |
| 32 | 30.1 | 31.9 |
| 33 | 0 | 40.5 |
| 34 | 0 | 39.5 |
| 35 | 0 | 36.6 |
| 36 | 0 | 37.5 |
| 37 | 0 | 38 |
| 38 | 6.4 | 38.6 |
| 39 | 0 | 39 |
| 40 | 0 | 38.3 |
| 41 | 0 | 42 |
| 42 | 0 | 39.4 |
| 43 | 0 | 41.6 |
| 44 | 0 | 41.3 |
| 45 | 0 | 39.1 |
| 46 | 0 | 39.1 |
| 47 | 0 | 39 |
| 48 | 0 | 38.2 |

TABLE 31
Monoplex

| Swab # | GC | AC |
|---|---|---|
| 49 | 331 | 26628 |
| 50 | 6344 | 23273 |
| 51 | 1217 | 30328 |
| 52 | 311 | 34605 |
| 53 | 196 | 33636 |
| 54 | 838 | 35845 |
| 55 | 590 | 40157 |
| 56 | 385 | 39401 |
| 57 | 418 | 35035 |
| 58 | 456 | 43324 |
| 59 | 22531 | 31816 |
| 60 | 295 | 37551 |
| 61 | 493 | 39241 |
| 62 | 835 | 36399 |
| 63 | 862 | 36163 |
| 64 | 533 | 26028 |
| 65 | 614 | 23176 |
| 66 | 525 | 24176 |
| 67 | 639 | 20922 |
| 68 | 440 | 29317 |
| 69 | 716 | 25602 |
| 70 | 535 | 27737 |
| 71 | 545 | 21254 |
| 72 | 501 | 19844 |

TABLE 32
Diplex

| Swab # | dGC | IAC |
|---|---|---|
| 49 | 0 | 40.9 |
| 50 | 0 | 37.8 |
| 51 | 0 | 42.8 |
| 52 | 0 | 39.8 |
| 53 | 0 | 40.2 |
| 54 | 0 | 39.8 |
| 55 | 0 | 39.9 |
| 56 | 0 | 39.7 |
| 57 | 0 | 37 |
| 58 | 0 | 38.9 |
| 59 | 0 | 35 |
| 60 | 0 | 36.5 |
| 61 | 0 | 39 |
| 62 | 0 | 35.8 |
| 63 | 0 | 34.4 |
| 64 | 0 | 36.3 |
| 65 | 0 | 38.3 |
| 66 | 0 | 40.7 |
| 67 | 0 | 40.9 |
| 68 | 0 | 41.1 |
| 69 | 0 | 42.3 |
| 70 | 0 | 39.7 |
| 71 | 0 | 39.9 |
| 72 | 0 | 36.9 |

TABLE 33
Monoplex

| Swab # | GC | AC |
|---|---|---|
| 73 | 277 | 58236 |
| 74 | 364 | 38829 |
| 75 | 8064 | 45206 |
| 76 | 887 | 48376 |
| 77 | 966 | 48913 |
| 78 | 330 | 51316 |
| 79 | 346 | 45505 |
| 80 | 442 | 51633 |
| 81 | 302 | 19278 |
| 82 | 259 | 19057 |
| 83 | 298 | 26156 |
| 84 | 322 | 24191 |
| 85 | 539 | 24740 |
| 86 | 588 | 26227 |
| 87 | 734 | 24115 |
| 88 | 386 | 16002 |
| 89 | 371 | 27913 |
| 90 | 558 | 33197 |
| 91 | 559 | 33815 |
| 92 | 778 | 30529 |
| 93 | 579 | 27800 |
| 94 | 636 | 35917 |
| 95 | 418 | 34602 |
| 96 | 363 | 25391 |

TABLE 34
Diplex

| Swab # | dGC | IAC |
|---|---|---|
| 73 | 0 | 36 |
| 74 | 0 | 39.8 |
| 75 | 0 | 37.9 |
| 76 | 0 | 40.6 |
| 77 | 0 | 40.5 |
| 78 | 0 | 40.3 |
| 79 | 0 | 42.6 |
| 80 | 0 | 42.4 |
| 81 | 0 | 41.2 |
| 82 | 0 | 37.5 |
| 83 | 0 | 40.4 |
| 84 | 0 | 39.9 |
| 85 | 0 | 39.9 |
| 86 | 0 | 40 |
| 87 | 0 | 41.2 |
| 88 | 0 | 39.5 |
| 89 | 0 | 36.2 |
| 90 | 0 | 36.7 |
| 91 | 0 | 38.1 |
| 92 | 0 | 36.6 |
| 93 | 0 | 38.2 |
| 94 | 0 | 34.8 |
| 95 | 0 | 34.5 |
| 96 | 0 | 36.9 |

### Example 3

### Day 3— Before Cleaning

All swabs are positive for GC in at least one assay (either monoplex or diplex). Therefore according to Av-17 Verification, the study can proceed to step 4.4.2.

TABLE 35
Monoplex

| Swab # | GC | AC |
|---|---|---|
| 1 | 28269 | 28675 |
| 2 | 28713 | 30598 |
| 3 | 32159 | 33354 |
| 4 | 25215 | 23263 |
| 5 | 27720 | 23861 |
| 6 | 30649 | 24231 |
| 7 | 30842 | 24298 |
| 8 | 25695 | 24114 |
| 9 | 26399 | 22461 |
| 10 | 25366 | 27534 |
| 11 | 26871 | 36559 |
| 12 | 19861 | 31157 |
| 13 | 17878 | 29555 |
| 14 | 24319 | 28074 |
| 15 | 25910 | 30255 |
| 16 | 20197 | 26469 |
| 17 | 24281 | 26253 |
| 18 | 30381 | 29170 |
| 19 | 30972 | 29832 |
| 20 | 29912 | 27532 |
| 21 | 30293 | 27264 |
| 22 | 27895 | 26693 |
| 23 | 28404 | 24931 |
| 24 | 25489 | 24125 |

TABLE 36
Diplex

| Swab # | dGC | IAC |
|---|---|---|
| 1 | 37.5 | 43 |
| 2 | 29.6 | 43.6 |
| 3 | 33.8 | 43.2 |
| 4 | 38.5 | 41.7 |
| 5 | 32.9 | 39.7 |
| 6 | 32.9 | 42.9 |
| 7 | 23 | 42.7 |
| 8 | 37.2 | 42.6 |
| 9 | 32.3 | 38.8 |
| 10 | 25.3 | 40.5 |
| 11 | 39.1 | 40.1 |
| 12 | 38 | 39.5 |
| 13 | 34.9 | 39.5 |
| 14 | 32 | 41 |
| 15 | 32.3 | 40.8 |
| 16 | 31.5 | 38.3 |
| 17 | 27.1 | 34.7 |
| 18 | 26.1 | 39.5 |
| 19 | 23 | 41.9 |
| 20 | 36.4 | 39.2 |
| 21 | 0 | 30.9 |
| 22 | 35.5 | 38.5 |
| 23 | 38.7 | 40 |
| 24 | 36.1 | 40.8 |

TABLE 37
Monoplex

| Swab # | GC | AC |
|---|---|---|
| 25 | 25359 | 32435 |
| 26 | 24834 | 34223 |
| 27 | 26929 | 35910 |
| 28 | 25380 | 37983 |
| 29 | 26948 | 36347 |
| 30 | 27467 | 31959 |
| 31 | 29334 | 39686 |
| 32 | 25386 | 35578 |
| 33 | 23573 | 19580 |
| 34 | 22706 | 22479 |
| 35 | 28262 | 26352 |
| 36 | 26692 | 24112 |
| 37 | 23947 | 27018 |
| 38 | 24597 | 26104 |
| 39 | 25836 | 32045 |
| 40 | 25751 | 29917 |
| 41 | 23622 | 22850 |
| 42 | 26037 | 22178 |
| 43 | 26189 | 23894 |
| 44 | 24838 | 19523 |
| 45 | 28887 | 23508 |
| 46 | 29770 | 23886 |
| 47 | 30308 | 24065 |
| 48 | 27980 | 24366 |

TABLE 38
Diplex

| Swab # | dGC | IAC |
|---|---|---|
| 25 | 33 | 38.9 |
| 26 | 32.3 | 35.7 |
| 27 | 36 | 31.6 |
| 28 | 33 | 40.1 |
| 29 | 35.4 | 41.9 |
| 30 | 30.9 | 39.9 |
| 31 | 33.2 | 39.4 |
| 32 | 37.1 | 39.3 |
| 33 | 28.3 | 42.1 |
| 34 | 28 | 40 |
| 35 | 32.8 | 36.1 |
| 36 | 32.8 | 42 |
| 37 | 31.4 | 42.4 |
| 38 | 28.3 | 37.6 |
| 39 | 33.1 | 40.3 |
| 40 | 38.1 | 38.5 |
| 41 | 31.1 | 41 |
| 42 | 18.1 | 37 |
| 43 | 37.1 | 40.5 |
| 44 | 29.9 | 40.4 |
| 45 | 31.5 | 40.8 |
| 46 | 9.3 | 39.9 |
| 47 | 35.2 | 41.4 |
| 48 | 36 | 42.5 |

TABLE 39
Monoplex

| Swab # | GC | AC |
|---|---|---|
| 49 | 20781 | 26675 |
| 50 | 23907 | 31573 |
| 51 | 20287 | 25482 |
| 52 | 20328 | 24307 |
| 53 | 20097 | 23162 |
| 54 | 21645 | 24400 |
| 55 | 22045 | 21234 |
| 56 | 23518 | 19612 |
| 57 | 27857 | 25814 |
| 58 | 29408 | 27977 |
| 59 | 28918 | 35343 |
| 60 | 27052 | 29925 |
| 61 | 30429 | 30729 |
| 62 | 31364 | 31792 |
| 63 | 30503 | 32883 |
| 64 | 30143 | 40039 |
| 65 | 31209 | 38709 |
| 66 | 35043 | 42193 |
| 67 | 31780 | 49960 |
| 68 | 26957 | 33457 |
| 69 | 30678 | 37343 |
| 70 | 31064 | 33598 |
| 71 | 28999 | 33832 |
| 72 | 24542 | 45846 |

TABLE 40
Diplex

| Swab # | dGC | IAC |
|---|---|---|
| 49 | 36.6 | 37.7 |
| 50 | 38.7 | 40.7 |
| 51 | 35.9 | 43.6 |
| 52 | 35.4 | 43.7 |
| 53 | 40.5 | 40.9 |
| 54 | 38.5 | 39.5 |
| 55 | 36.7 | 43.9 |
| 56 | 14.1 | 41.5 |
| 57 | 32.2 | 42.4 |
| 58 | 35.5 | 37.9 |
| 59 | 34.6 | 36.4 |
| 60 | 29.6 | 39.1 |
| 61 | 37.4 | 39.4 |
| 62 | 38.4 | 34.7 |
| 63 | 23.3 | 40.4 |
| 64 | 37.5 | 40.3 |
| 65 | 29.8 | 39.9 |
| 66 | 22.9 | 37.9 |
| 67 | 36.3 | 36.4 |
| 68 | 34.7 | 39.1 |
| 69 | 34.1 | 39.4 |
| 70 | 36.7 | 34.7 |
| 71 | 28.2 | 40.4 |
| 72 | 32.7 | 43.6 |

TABLE 41
Monoplex

| Swab # | GC | AC |
|---|---|---|
| 73 | 28107 | 35932 |
| 74 | 31822 | 30339 |
| 75 | 35960 | 32628 |
| 76 | 28681 | 31094 |
| 77 | 28334 | 37460 |
| 78 | 32436 | 43941 |
| 79 | 29070 | 47051 |
| 80 | 26192 | 33008 |
| 81 | 21642 | 39498 |
| 82 | 24427 | 40681 |
| 83 | 17199 | 33291 |
| 84 | 21546 | 49143 |
| 85 | 23140 | 60859 |
| 86 | 19817 | 51050 |
| 87 | 23894 | 32179 |
| 88 | 25042 | 55674 |
| 89 | 19735 | 35744 |
| 90 | 29270 | 24251 |
| 91 | 22414 | 28916 |
| 92 | 34374 | 33019 |
| 93 | 32129 | 36949 |
| 94 | 29136 | 40456 |
| 95 | 34883 | 42202 |
| 96 | 38119 | 36930 |

TABLE 42
Diplex

| Swab # | dGC | IAC |
|---|---|---|
| 73 | 33.5 | 42.1 |
| 74 | 29.6 | 39.9 |
| 75 | 25.6 | 42.8 |
| 76 | 36.7 | 38.7 |
| 77 | 36.7 | 36.9 |
| 78 | 31.8 | 38.7 |
| 79 | 9.5 | 38.9 |
| 80 | 34.7 | 42.7 |
| 81 | 28.1 | 43.8 |
| 82 | 35.8 | 42.3 |
| 83 | 27.9 | 44.6 |
| 84 | 38.5 | 38.8 |
| 85 | 29.1 | 42.8 |
| 86 | 32 | 43.6 |
| 87 | 18 | 43.3 |
| 88 | 35.9 | 41 |
| 89 | 38.3 | 42.8 |
| 90 | 38.2 | 40.3 |
| 91 | 38.6 | 43.2 |
| 92 | 25.6 | 43.6 |
| 93 | 34.8 | 42.2 |
| 94 | 36.5 | 43.4 |
| 95 | 37.9 | 42.1 |
| 96 | 34.9 | 41.5 |

### Example 3

### Day 3— After Cleaning

Comments:

78/96 swabs tested negative in both assays after cleaning the surface with hydrogen peroxide resulting in a reduction rate of 81%.

Dilute Hydrogen Peroxide was liberally poured onto the 48 contaminated squares, allowed to stand for 3 minutes, then wiped away in a one-directional motion. These steps were repeated and then 2 swabs were taken from each square, processed and tested.

Positive readings=Monoplex>2000; Diplex>0

TABLE 43
Monoplex

| Swab # | GC | AC |
|---|---|---|
| 1 | 5751 | 39114 |
| 2 | 25 | 49599 |
| 3 | 299 | 37472 |
| 4 | 203 | 40416 |
| 5 | 17215 | 34549 |
| 6 | 293 | 38176 |
| 7 | 180 | 47261 |
| 8 | 308 | 34214 |
| 9 | 2007 | 37580 |
| 10 | 132 | 37678 |
| 11 | 257 | 40897 |
| 12 | 250 | 41077 |
| 13 | 589 | 44152 |
| 14 | 530 | 51009 |
| 15 | 470 | 43644 |
| 16 | 526 | 40733 |
| 17 | 192 | 40000 |
| 18 | 324 | 39620 |
| 19 | 561 | 46059 |
| 20 | 319 | 39300 |
| 21 | 327 | 55292 |
| 22 | 341 | 2553 |
| 23 | 737 | 50389 |
| 24 | 592 | 36519 |

TABLE 44
Diplex

| Swab # | dGC | IAC |
|---|---|---|
| 1 | 0 | 43.5 |
| 2 | 0 | 43 |
| 3 | 0 | 43.9 |
| 4 | 0 | 42.6 |
| 5 | 0 | 39.4 |
| 6 | 0 | 42.3 |
| 7 | 0 | 43.1 |
| 8 | 0 | 43.1 |
| 9 | 0 | 39.6 |
| 10 | 0 | 44.2 |
| 11 | 0 | 42.4 |
| 12 | 0 | 41.9 |
| 13 | 0 | 44.3 |
| 14 | 0 | 43.7 |
| 15 | 0 | 41.3 |
| 16 | 0 | 44.4 |
| 17 | 0 | 42 |
| 18 | 0 | 41.4 |
| 19 | 0 | 41.4 |
| 20 | 0 | 41.9 |
| 21 | 0 | 42.2 |
| 22 | 0 | 41.3 |
| 23 | 0 | 42.5 |
| 24 | 0 | 44.1 |

TABLE 45
Monoplex

| Swab # | GC | AC |
|---|---|---|
| 25 | 294 | 45861 |
| 26 | 12726 | 36094 |
| 27 | 540 | 40088 |
| 28 | 357 | 37801 |
| 29 | 551 | 43124 |
| 30 | 686 | 45622 |
| 31 | 577 | 49116 |
| 32 | 323 | 40229 |
| 33 | 198 | 57227 |
| 34 | 516 | 46376 |
| 35 | 25 | 50839 |
| 36 | 157 | 37183 |
| 37 | 751 | 40349 |
| 38 | 559 | 47310 |
| 39 | 523 | 33604 |
| 40 | 681 | 36136 |
| 41 | 262 | 33835 |
| 42 | 460 | 31611 |
| 43 | 666 | 32606 |
| 44 | 244 | 32778 |
| 45 | 298 | 36770 |
| 46 | 287 | 28718 |
| 47 | 3279 | 30035 |
| 48 | 731 | 28123 |

TABLE 46
Diplex

| Swab # | dGC | IAC |
|---|---|---|
| 25 | 0 | 41.6 |
| 26 | 0 | 41.5 |
| 27 | 0 | 40.3 |
| 28 | 0 | 43 |
| 29 | 0 | 42.6 |
| 30 | 0 | 42.5 |
| 31 | 0 | 41.7 |
| 32 | 0 | 41.9 |
| 33 | 0 | 41.9 |
| 34 | 0 | 42.6 |
| 35 | 0 | 42.4 |
| 36 | 0 | 42.2 |
| 37 | 0 | 41.3 |
| 38 | 0 | 43.1 |
| 39 | 0 | 42 |
| 40 | 0 | 39.6 |
| 41 | 0 | 39.4 |
| 42 | 0 | 41.2 |
| 43 | 0 | 41.5 |
| 44 | 0 | 42.2 |
| 45 | 0 | 41.8 |
| 46 | 0 | 40.4 |
| 47 | 0 | 42 |
| 48 | 0 | 40.8 |

TABLE 47
Monoplex

| Swab # | GC | AC |
|---|---|---|
| 49 | 273 | 42131 |
| 50 | 594 | 61642 |
| 51 | 1114 | 34843 |
| 52 | 1097 | 46640 |
| 53 | 1042 | 42126 |
| 54 | 13659 | 46112 |
| 55 | 1034 | 38427 |
| 56 | 527 | 40274 |
| 57 | 183 | 48220 |
| 58 | 500 | 38077 |
| 59 | 721 | 46726 |
| 60 | 993 | 45463 |
| 61 | 1276 | 37482 |
| 62 | 451 | 46061 |
| 63 | 430 | 42818 |
| 64 | 821 | 28208 |
| 65 | 2525 | 52564 |
| 66 | 932 | 50197 |
| 67 | 24980 | 41297 |
| 68 | 1324 | 43962 |
| 69 | 584 | 50562 |
| 70 | 409 | 43029 |
| 71 | 577 | 51491 |
| 72 | 382 | 40703 |

TABLE 48
Diplex

| Swab # | dGC | IAC |
|---|---|---|
| 49 | 0 | 37.6 |
| 50 | 0 | 38.9 |
| 51 | 0 | 41.1 |
| 52 | 0 | 41.8 |
| 53 | 0 | 42.6 |
| 54 | 0 | 40.3 |
| 55 | 0 | 39.2 |
| 56 | 0 | 43.5 |
| 57 | 0 | 40.5 |
| 58 | 0 | 40.4 |
| 59 | 18.2 | 34.3 |
| 60 | 0 | 38.7 |
| 61 | 0 | 35.8 |
| 62 | 0 | 37.3 |
| 63 | 0 | 39 |
| 64 | 0 | 35.4 |
| 65 | 0 | 39.7 |
| 66 | 0 | 39.3 |
| 67 | 0 | 38.8 |
| 68 | 0 | 39 |
| 69 | 0 | 38.5 |
| 70 | 0 | 33.2 |
| 71 | 0 | 36 |
| 72 | 0 | 35 |

TABLE 49
Monoplex

| Swab # | GC | AC |
|---|---|---|
| 73 | 17860 | 41914 |
| 74 | 88 | 42132 |
| 75 | 3162 | 45307 |
| 76 | 8392 | 47415 |
| 77 | 935 | 37677 |
| 78 | 661 | 43790 |
| 79 | 774 | 44761 |
| 80 | 24303 | 31399 |
| 81 | 21888 | 38229 |
| 82 | 540 | 40997 |
| 83 | 634 | 42981 |
| 84 | 695 | 33747 |
| 85 | 3217 | 37516 |
| 86 | 742 | 37621 |
| 87 | 938 | 29094 |
| 88 | 1339 | 24077 |
| 89 | 14100 | 33408 |
| 90 | 33396 | 45378 |
| 91 | 33304 | 40631 |
| 92 | 765 | 40744 |
| 93 | 897 | 36348 |
| 94 | 1591 | 40470 |
| 95 | 13982 | 40999 |
| 96 | 771 | 38647 |

TABLE 50
Diplex

| Swab # | dGC | IAC |
|---|---|---|
| 73 | 0 | 38.8 |
| 74 | 0 | 36.6 |
| 75 | 0 | 39.8 |
| 76 | 0 | 35.6 |
| 77 | 0 | 37.2 |
| 78 | 0 | 37.1 |
| 79 | 0 | 35.5 |
| 80 | 0 | 38 |
| 81 | 0 | 38.2 |
| 82 | 0 | 37.8 |
| 83 | 0 | 41.4 |
| 84 | 0 | 42.7 |
| 85 | 12.4 | 41.3 |
| 86 | 0 | 42.9 |
| 87 | 0 | 39.9 |
| 88 | 0 | 43.3 |
| 89 | 0 | 37 |
| 90 | 0 | 39.8 |
| 91 | 0 | 40.6 |
| 92 | 0 | 39.6 |
| 93 | 0 | 41.4 |
| 94 | 0 | 39.5 |
| 95 | 0 | 40.3 |
| 96 | 0 | 39.2 |

### Example 3

### Day 5— Before Cleaning

All swabs are positive for GC in at least one assay (either monoplex or diplex). Therefore according to AV-17 Verification, the stud can proceed to step 4.4.2.

TABLE 51
Monoplex

| Swab # | GC | AC |
|---|---|---|
| 1 | 23009 | 40562 |
| 2 | 26577 | 40316 |
| 3 | 29876 | 38775 |
| 4 | 22300 | 33885 |
| 5 | 27645 | 38243 |
| 6 | 19933 | 35072 |
| 7 | 19117 | 37660 |
| 8 | 16265 | 34828 |
| 9 | 26453 | 42292 |
| 10 | 28105 | 39623 |
| 11 | 30862 | 47444 |
| 12 | 31149 | 47756 |
| 13 | 29971 | 48294 |
| 14 | 25929 | 42454 |
| 15 | 26655 | 39936 |
| 16 | 25358 | 52489 |
| 17 | 22344 | 47246 |
| 18 | 30948 | 34252 |
| 19 | 36560 | 45075 |
| 20 | 30724 | 37856 |
| 21 | 35273 | 42456 |
| 22 | 32790 | 40457 |
| 23 | 34967 | 37327 |
| 24 | 27614 | 45958 |

TABLE 52
Diplex

| Swab # | dGC | IAC |
|---|---|---|
| 1 | 29.7 | 31.2 |
| 2 | 31.2 | 37.3 |
| 3 | 35.5 | 36.8 |
| 4 | 37.7 | 38.9 |
| 5 | 33.7 | 39.1 |
| 6 | 21.1 | 41.4 |
| 7 | 37.3 | 42.8 |
| 8 | 32.6 | 42.2 |
| 9 | 34.4 | 39.5 |
| 10 | 14.5 | 43.1 |
| 11 | 36 | 40.4 |
| 12 | 32.2 | 40.1 |
| 13 | 20.8 | 39 |
| 14 | 21.9 | 40.2 |
| 15 | 26.8 | 40.6 |
| 16 | 33.3 | 37.9 |
| 17 | 22.3 | 33.2 |
| 18 | 29.2 | 39.9 |
| 19 | 28.6 | 36.9 |
| 20 | 33.1 | 36.7 |
| 21 | 32.2 | 37.8 |
| 22 | 34.8 | 35.8 |
| 23 | 31.4 | 38.5 |
| 24 | 13.6 | 40.6 |

TABLE 53
Monoplex

| Swab # | GC | AC |
|---|---|---|
| 25 | 29201 | 41108 |
| 26 | 40739 | 53646 |
| 27 | 31426 | 46343 |
| 28 | 28727 | 34127 |
| 29 | 28614 | 35006 |
| 30 | 34448 | 38076 |
| 31 | 29779 | 30007 |
| 32 | 28336 | 22292 |
| 33 | 29823 | 44407 |
| 34 | 30570 | 49599 |
| 35 | 32082 | 40517 |
| 36 | 26663 | 57011 |
| 37 | 30479 | 53485 |
| 38 | 29013 | 45452 |
| 39 | 27707 | 43117 |
| 40 | 29377 | 40858 |
| 41 | 26571 | 46937 |
| 42 | 33402 | 39512 |
| 43 | 31163 | 41068 |
| 44 | 30759 | 37567 |
| 45 | 28251 | 37001 |
| 46 | 28432 | 40295 |
| 47 | 33074 | 42755 |
| 48 | 24408 | 26841 |

TABLE 54
Diplex

| Swab # | dGC | IAC |
|---|---|---|
| 25 | 14.4 | 43.3 |
| 26 | 20.7 | 38.8 |
| 27 | 19.6 | 42.2 |
| 28 | 24.8 | 40.2 |
| 29 | 24 | 41.7 |
| 30 | 23.3 | 42.4 |
| 31 | 30.4 | 41.1 |
| 32 | 30.7 | 41.6 |
| 33 | 29 | 29.5 |
| 34 | 12.8 | 37.9 |
| 35 | 28.2 | 38.2 |
| 36 | 28.7 | 40 |
| 37 | 34.8 | 38.7 |
| 38 | 29.9 | 38.4 |
| 39 | 32.2 | 37.9 |
| 40 | 32.3 | 38.2 |
| 41 | 35.7 | 29.9 |
| 42 | 32.3 | 38.5 |
| 43 | 29.2 | 38.5 |
| 44 | 33.9 | 37 |
| 45 | 31.9 | 40 |
| 46 | 30.3 | 38.2 |
| 47 | 34.8 | 38.3 |
| 48 | 34.9 | 33.2 |

TABLE 55
Monoplex

| Swab # | GC | AC |
|---|---|---|
| 49 | 25094 | 33676 |
| 50 | 31386 | 44991 |
| 51 | 29830 | 48186 |
| 52 | 37189 | 51529 |
| 53 | 37999 | 56120 |
| 54 | 26350 | 45854 |
| 55 | 33093 | 39654 |
| 56 | 29205 | 33619 |
| 57 | 20193 | 25575 |
| 58 | 28386 | 40288 |
| 59 | 24537 | 37120 |
| 60 | 26192 | 40023 |
| 61 | 22203 | 34134 |
| 62 | 28671 | 44278 |
| 63 | 23859 | 34430 |
| 64 | 21967 | 23673 |
| 65 | 25512 | 41513 |
| 66 | 28647 | 49577 |
| 67 | 31960 | 38860 |
| 68 | 32386 | 45532 |
| 69 | 26973 | 37080 |
| 70 | 27483 | 47566 |
| 71 | 31456 | 46548 |
| 72 | 24671 | 36217 |

TABLE 56
Diplex

| Swab # | dGC | IAC |
|---|---|---|
| 49 | 0 | 40.5 |
| 50 | 14.3 | 41.1 |
| 51 | 4.2 | 41 |
| 52 | 0 | 37.9 |
| 53 | 20 | 40.4 |
| 54 | 23 | 42 |
| 55 | 34.1 | 41 |
| 56 | 23.2 | 39.8 |
| 57 | 0 | 34.7 |
| 58 | 25.8 | 29.6 |
| 59 | 0 | 40.5 |
| 60 | 34.8 | 35.2 |
| 61 | 0 | 39 |
| 62 | 0 | 37.2 |
| 63 | 0 | 40.2 |
| 64 | 0 | 39 |
| 65 | 0 | 41 |
| 66 | 0 | 38.1 |
| 67 | 12 | 35.7 |
| 68 | 4.1 | 40.1 |
| 69 | 30.6 | 39.3 |
| 70 | 15.2 | 42.4 |
| 71 | 3.8 | 44.4 |
| 72 | 5.9 | 41 |

TABLE 57
Monoplex

| Swab # | GC | AC |
|---|---|---|
| 73 | 30106 | 15578 |
| 74 | 31548 | 40262 |
| 75 | 29591 | 48258 |
| 76 | 41748 | 38891 |
| 77 | 39939 | 25659 |
| 78 | 32962 | 56889 |
| 79 | 29348 | 56762 |
| 80 | 30145 | 15697 |
| 81 | 25405 | 51494 |
| 82 | 28531 | 54127 |
| 83 | 30792 | 49439 |
| 84 | 29030 | 58478 |
| 85 | 31400 | 50504 |
| 86 | 33641 | 52312 |
| 87 | 26232 | 34899 |
| 88 | 25001 | 26889 |
| 89 | 25151 | 45733 |
| 90 | 39127 | 54393 |
| 91 | 25204 | 38415 |
| 92 | 29510 | 57016 |
| 93 | 28365 | 44002 |
| 94 | 27723 | 54297 |
| 95 | 30073 | 40586 |
| 96 | 24243 | 35190 |

TABLE 58
Diplex

| Swab # | dGC | IAC |
|---|---|---|
| 73 | 13.7 | 38.6 |
| 74 | 0 | 39.9 |
| 75 | 27.8 | 39.3 |
| 76 | 11.4 | 37.7 |
| 77 | 29.2 | 38.3 |
| 78 | 28.3 | 41.6 |
| 79 | 33.2 | 36.2 |
| 80 | 29 | 41.1 |
| 81 | 33.5 | 31.9 |
| 82 | 33.5 | 35.2 |
| 83 | 28.8 | 37.1 |
| 84 | 33.3 | 38.9 |
| 85 | 36.6 | 41.4 |
| 86 | 34.5 | 38.8 |
| 87 | 35.8 | 40.8 |
| 88 | 38.5 | 34.1 |
| 89 | 36 | 34.6 |
| 90 | 33.1 | 41.2 |
| 91 | 37.1 | 31 |
| 92 | 36.8 | 27.8 |
| 93 | 30.7 | 41.2 |
| 94 | 33.9 | 40.4 |
| 95 | 33.6 | 39.5 |
| 96 | 38.1 | 38.7 |

### Example 3

### Day 5— After Cleaning

Comments:

84/96 swabs tested negative in both assays after cleaning the surface with hydrogen peroxide resulting in a reduction rate of 88%.

Dilute Hydrogen Peroxide was liberally poured onto the 48 contaminated squares, allowed to stand for 3 minutes, then wiped away in a one-directional motion. These steps were repeated and then 2 swabs were taken from each square, processed and tested.

Positive readings=Monoplex>2000; Diplex>0

TABLE 59
Monoplex

| Swab # | GC | AC |
|---|---|---|
| 1 | 9619 | 33237 |
| 2 | 472 | 52156 |
| 3 | 462 | 41883 |
| 4 | 405 | 45453 |
| 5 | 420 | 38909 |
| 6 | 451 | 41785 |
| 7 | 449 | 40829 |
| 8 | 443 | 33016 |
| 9 | 429 | 47621 |
| 10 | 440 | 44767 |
| 11 | 383 | 59993 |
| 12 | 339 | 45096 |
| 13 | 239 | 52275 |
| 14 | 360 | 50065 |
| 15 | 10 | 44358 |
| 16 | 336 | 42785 |
| 17 | 469 | 32193 |
| 18 | 541 | 30172 |
| 19 | 311 | 33607 |
| 20 | 454 | 34474 |
| 21 | 289 | 40475 |
| 22 | 396 | 31906 |
| 23 | 308 | 32182 |
| 24 | 280 | 33715 |

TABLE 60
Diplex

| Swab # | dGC | IAC |
|---|---|---|
| 1 | 0 | 44.6 |
| 2 | 0 | 42.6 |
| 3 | 0 | 43.1 |
| 4 | 0 | 42 |
| 5 | 0 | 42 |
| 6 | 0 | 41.5 |
| 7 | 0 | 42.3 |
| 8 | 0 | 39.2 |
| 9 | 0 | 42.9 |
| 10 | 0 | 42.7 |
| 11 | 0 | 43 |
| 12 | 0 | 43.4 |
| 13 | 0 | 41.8 |
| 14 | 0 | 41.8 |
| 15 | 0 | 42.6 |
| 16 | 0 | 42.4 |
| 17 | 0 | 43.5 |
| 18 | 0 | 39.9 |
| 19 | 0 | 43 |
| 20 | 0 | 42.8 |
| 21 | 0 | 42.8 |
| 22 | 0 | 41.2 |
| 23 | 0 | 41.8 |
| 24 | 0 | 41.4 |

TABLE 61
Monoplex

| Swab # | GC | AC |
|---|---|---|
| 25 | 406 | 43661 |
| 26 | 688 | 45628 |
| 27 | 364 | 51911 |
| 28 | 412 | 51949 |
| 29 | 602 | 41483 |
| 30 | 612 | 41653 |
| 31 | 359 | 51002 |
| 32 | 337 | 43837 |
| 33 | 464 | 55289 |
| 34 | 652 | 56726 |
| 35 | 828 | 49175 |
| 36 | 574 | 53555 |
| 37 | 444 | 53683 |
| 38 | 717 | 40472 |
| 39 | 415 | 38670 |
| 40 | 411 | 35603 |
| 41 | 1363 | 34021 |
| 42 | 401 | 34518 |
| 43 | 11094 | 29699 |
| 44 | 768 | 35252 |
| 45 | 11945 | 37411 |
| 46 | 24287 | 35033 |
| 47 | 741 | 39330 |
| 48 | 365 | 32297 |

TABLE 62
Diplex

| Swab # | dGC | IAC |
|---|---|---|
| 25 | 0 | 42.9 |
| 26 | 0 | 42.3 |
| 27 | 0 | 43.7 |
| 28 | 0 | 42 |
| 29 | 0 | 41.3 |
| 30 | 0 | 43.3 |
| 31 | 0 | 41.5 |
| 32 | 0 | 39.7 |
| 33 | 0 | 43.1 |
| 34 | 0 | 41.1 |
| 35 | 0 | 42.7 |
| 36 | 0 | 41.5 |
| 37 | 0 | 39.6 |
| 38 | 0 | 40.8 |
| 39 | 0 | 42.5 |
| 40 | 0 | 40.9 |
| 41 | 0 | 42.3 |
| 42 | 0 | 42.5 |
| 43 | 0 | 41.4 |
| 44 | 0 | 42.3 |
| 45 | 0 | 42.1 |
| 46 | 0 | 39.3 |
| 47 | 0 | 40.9 |
| 48 | 0 | 41.2 |

TABLE 63
Monoplex

| Swab # | GC | AC |
|---|---|---|
| 49 | 301 | 44083 |
| 50 | 78 | 58387 |
| 51 | 148 | 42208 |
| 52 | 117 | 52129 |
| 53 | 50 | 59421 |
| 54 | 77 | 52677 |
| 55 | 171 | 57628 |
| 56 | 351 | 48090 |
| 57 | 821 | 42518 |
| 58 | 527 | 56048 |
| 59 | 612 | 64727 |
| 60 | 330 | 56105 |
| 61 | 769 | 56571 |
| 62 | 840 | 47920 |
| 63 | 333 | 40764 |
| 64 | 778 | 27526 |
| 65 | 419 | 45466 |
| 66 | 509 | 44726 |
| 67 | 865 | 35466 |
| 68 | 806 | 46479 |
| 69 | 1008 | 56753 |
| 70 | 5148 | 44455 |
| 71 | 338 | 55593 |
| 72 | 604 | 32514 |

TABLE 64
Diplex

| Swab # | dGC | IAC |
|---|---|---|
| 49 | 0 | 41 |
| 50 | 0 | 33.7 |
| 51 | 0 | 42 |
| 52 | 0 | 41.7 |
| 53 | 0 | 38.8 |
| 54 | 0 | 38.8 |
| 55 | 0 | 41 |
| 56 | 0 | 29.3 |
| 57 | 0 | 43.7 |
| 58 | 0 | 43.7 |
| 59 | 0 | 43.9 |
| 60 | 0 | 43.2 |
| 61 | 0 | 42.8 |
| 62 | 0 | 44.4 |
| 63 | 0 | 41.9 |
| 64 | 0 | 25.5 |
| 65 | 0 | 40.9 |
| 66 | 0 | 41 |
| 67 | 0 | 43.5 |
| 68 | 0 | 40.5 |
| 69 | 0 | 43.8 |
| 70 | 0 | 39.8 |
| 71 | 0 | 35.6 |
| 72 | 0 | 32 |

TABLE 65
Monoplex

| Swab # | GC | AC |
|---|---|---|
| 73 | 1010 | 53281 |
| 74 | 623 | 59035 |
| 75 | 5170 | 39881 |
| 76 | 263 | 46845 |
| 77 | 523 | 56542 |
| 78 | 264 | 53710 |
| 79 | 347 | 53738 |
| 80 | 745 | 35921 |
| 81 | 352 | 40240 |
| 82 | 398 | 44022 |
| 83 | 585 | 55182 |
| 84 | 681 | 47458 |
| 85 | 2716 | 41183 |
| 86 | 1032 | 38811 |
| 87 | 1139 | 45937 |
| 88 | 547 | 43834 |
| 89 | 440 | 23338 |
| 90 | 745 | 24383 |
| 91 | 561 | 31253 |
| 92 | 547 | 31604 |
| 93 | 588 | 34870 |
| 94 | 796 | 33059 |
| 95 | 831 | 29424 |
| 96 | 411 | 27360 |

TABLE 66
Diplex

| Swab # | dGC | IAC |
|---|---|---|
| 73 | 0 | 39.3 |
| 74 | 0 | 23.7 |
| 75 | 0 | 34.6 |
| 76 | 20.5 | 14.1 |
| 77 | 0 | 40.7 |
| 78 | 0 | 37.9 |
| 79 | 32.3 | 39 |
| 80 | 0 | 36 |
| 81 | 0 | 38.9 |
| 82 | 0 | 41.2 |
| 83 | 0 | 44.2 |
| 84 | 15.1 | 22.9 |
| 85 | 0 | 40.9 |
| 86 | 0 | 41.9 |
| 87 | 0 | 41.4 |
| 88 | 0 | 40.5 |
| 89 | 13.9 | 42.6 |
| 90 | 0 | 39.3 |
| 91 | 0 | 29.3 |
| 92 | 15.9 | 41.7 |
| 93 | 0 | 41.9 |
| 94 | 0 | 40.8 |
| 95 | 0 | 39.1 |
| 96 | 0 | 40.9 |

### Example 3

### Day 8—Before Cleaning

All swabs are positive for GC in at least one assay (either monoplex or diplex). Therefore according to AV-17 Verification, the study can proceed to step 4.4.2.

TABLE 67
Monoplex

| Swab # | GC | AC |
|---|---|---|
| 1 | 34218 | 28572 |
| 2 | 34731 | 37477 |
| 3 | 33560 | 28999 |
| 4 | 32114 | 28145 |
| 5 | 32970 | 28804 |
| 6 | 27705 | 23330 |
| 7 | 25646 | 26112 |
| 8 | 22720 | 30697 |
| 9 | 24061 | 52874 |
| 10 | 23719 | 42203 |
| 11 | 26768 | 54382 |
| 12 | 24246 | 33472 |
| 13 | 24920 | 35321 |
| 14 | 22810 | 32819 |
| 15 | 23472 | 31461 |
| 16 | 24918 | 32090 |
| 17 | 24962 | 33355 |
| 18 | 25639 | 39502 |
| 19 | 31440 | 43734 |
| 20 | 25346 | 31280 |
| 21 | 27893 | 40488 |
| 22 | 25817 | 35960 |
| 23 | 29259 | 31616 |
| 24 | 25597 | 34019 |

TABLE 68
Diplex

| Swab # | dGC | IAC |
|---|---|---|
| 1 | 33.3 | 38.5 |
| 2 | 39.5 | 35.5 |
| 3 | 37.6 | 38.5 |
| 4 | 17.6 | 41.1 |
| 5 | 39.5 | 31.8 |
| 6 | 35.7 | 37.5 |
| 7 | 36.1 | 36.7 |
| 8 | 33.8 | 38.9 |
| 9 | 34.9 | 36.1 |
| 10 | 36.5 | 40.5 |
| 11 | 39.3 | 35.5 |
| 12 | 0 | 41 |
| 13 | 37.3 | 37.4 |
| 14 | 35.9 | 37 |
| 15 | 36.7 | 33 |
| 16 | 32.2 | 37.8 |
| 17 | 8.8 | 39.2 |
| 18 | 0 | 43.9 |
| 19 | 41.5 | 38.2 |
| 20 | 0 | 43.6 |
| 21 | 38.5 | 37.2 |
| 22 | 37.1 | 43.8 |
| 23 | 40.7 | 37.3 |
| 24 | 38.1 | 32.8 |

TABLE 69
Monoplex

| Swab # | GC | AC |
|---|---|---|
| 25 | 28829 | 35884 |
| 26 | 29125 | 48436 |
| 27 | 30107 | 40476 |
| 28 | 27001 | 40800 |
| 29 | 28841 | 45752 |
| 30 | 26529 | 32369 |
| 31 | 28649 | 38714 |
| 32 | 26641 | 25595 |
| 33 | 24195 | 49374 |
| 34 | 27538 | 47288 |
| 35 | 29941 | 43319 |
| 36 | 26906 | 50361 |
| 37 | 28763 | 46044 |
| 38 | 25489 | 40462 |
| 39 | 30482 | 46252 |
| 40 | 22100 | 42740 |
| 41 | 25416 | 22918 |
| 42 | 28381 | 41175 |
| 43 | 25012 | 33289 |
| 44 | 28297 | 41713 |
| 45 | 29961 | 33447 |
| 46 | 33681 | 39634 |
| 47 | 29400 | 45541 |
| 48 | 25889 | 40960 |

TABLE 70
Diplex

| Swab # | dGC | IAC |
|---|---|---|
| 25 | 39.6 | 38.7 |
| 26 | 37.6 | 43.2 |
| 27 | 0 | 0 |
| 28 | 38.6 | 39.3 |
| 29 | 38.7 | 36.2 |
| 30 | 39 | 39.5 |
| 31 | 39.2 | 35.4 |
| 32 | 38.4 | 32.6 |
| 33 | 34 | 40.1 |
| 34 | 37.6 | 41 |
| 35 | 0 | 13.3 |
| 36 | 35.8 | 33.6 |
| 37 | 38.9 | 40.3 |
| 38 | 34.7 | 41.3 |
| 39 | 27.7 | 22.5 |
| 40 | 37.9 | 34.5 |
| 41 | 33.7 | 32.3 |
| 42 | 24.1 | 36.3 |
| 43 | 32.5 | 23.6 |
| 44 | 34.4 | 30.6 |
| 45 | 33.2 | 28.7 |
| 46 | 24.2 | 22 |
| 47 | 32.3 | 34.8 |
| 48 | 10.6 | 30.6 |

TABLE 71
Monoplex

| Swab # | GC | AC |
|---|---|---|
| 49 | 27932 | 37044 |
| 50 | 28515 | 48930 |
| 51 | 29920 | 44381 |
| 52 | 28790 | 47069 |
| 53 | 25462 | 38026 |
| 54 | 28008 | 46635 |
| 55 | 32136 | 43514 |
| 56 | 27957 | 47381 |
| 57 | 27346 | 30722 |
| 58 | 33807 | 45084 |
| 59 | 30774 | 32495 |
| 60 | 31358 | 30388 |
| 61 | 30820 | 51444 |
| 62 | 28517 | 46950 |
| 63 | 29792 | 39421 |
| 64 | 26771 | 41193 |
| 65 | 26094 | 43221 |
| 66 | 29721 | 47409 |
| 67 | 27028 | 59023 |
| 68 | 30286 | 46546 |
| 69 | 28818 | 51287 |
| 70 | 24318 | 31282 |
| 71 | 24340 | 31622 |
| 72 | 19194 | 28583 |

TABLE 72
Diplex

| Swab # | dGC | IAC |
|---|---|---|
| 49 | 31.5 | 39.2 |
| 50 | 36 | 36.1 |
| 51 | 33.6 | 37.9 |
| 52 | 35.4 | 37.7 |
| 53 | 24.4 | 36.6 |
| 54 | 31.6 | 36.3 |
| 55 | 37 | 30.8 |
| 56 | 21.2 | 30.8 |
| 57 | 36.4 | 34.4 |
| 58 | 35.6 | 30.5 |
| 59 | 36 | 34 |
| 60 | 33.9 | 30.7 |
| 61 | 31.2 | 35.4 |
| 62 | 35.9 | 32.5 |
| 63 | 34.6 | 36.8 |
| 64 | 35.8 | 30.8 |
| 65 | 28.7 | 27.9 |
| 66 | 32.5 | 28 |
| 67 | 31.1 | 33.7 |
| 68 | 32.6 | 31.3 |
| 69 | 16.4 | 28.4 |
| 70 | 19.2 | 23.2 |
| 71 | 32.3 | 33.7 |
| 72 | 21.4 | 31.3 |

TABLE 73
Monoplex

| Swab # | GC | AC |
|---|---|---|
| 73 | 26529 | 39221 |
| 74 | 28321 | 50860 |
| 75 | 32996 | 41992 |
| 76 | 33774 | 54266 |
| 77 | 33622 | 39613 |
| 78 | 36040 | 43275 |
| 79 | 31719 | 54146 |
| 80 | 29156 | 39048 |
| 81 | 17727 | 24910 |
| 82 | 17989 | 26189 |
| 83 | 19460 | 39300 |
| 84 | 19777 | 29916 |
| 85 | 19545 | 36606 |
| 86 | 23663 | 30455 |
| 87 | 22504 | 34760 |
| 88 | 21937 | 38570 |
| 89 | 22723 | 16069 |
| 90 | 27638 | 17416 |
| 91 | 26043 | 25689 |
| 92 | 19192 | 20371 |
| 93 | 18370 | 17640 |
| 94 | 20528 | 16623 |
| 95 | 18283 | 19858 |
| 96 | 17648 | 17270 |

TABLE 74
Diplex

| Swab # | dGC | IAC |
|---|---|---|
| 73 | 11 | 14.6 |
| 74 | 36.9 | 27 |
| 75 | 26.8 | 30.6 |
| 76 | 29.1 | 26.5 |
| 77 | 37 | 20.7 |
| 78 | 35.2 | 31.6 |
| 79 | 23.9 | 29.8 |
| 80 | 25.9 | 35.5 |
| 81 | 37.2 | 35.8 |
| 82 | 36.3 | 34.7 |
| 83 | 21.5 | 39.7 |
| 84 | 19.4 | 22.2 |
| 85 | 25.9 | 30.3 |
| 86 | 34.7 | 35.3 |
| 87 | 15.7 | 26.2 |
| 88 | 38.9 | 25.8 |
| 89 | 35.2 | 34 |
| 90 | 28.5 | 32.1 |
| 91 | 31.4 | 34.2 |
| 92 | 26.4 | 38.5 |
| 93 | 0 | 41.3 |
| 94 | 0 | 39.5 |
| 95 | 31.1 | 11.5 |
| 96 | 33.2 | 29.2 |

### Example 3

### Day 8—After Cleaning

Comments:

78/96 swabs tested negative in both assays after cleaning the surface with hydrogen peroxide resulting in a reduction rate of 81%.

Hydrogen Peroxide was liberally poured onto the 48 contaminated squares, allowed to stand for 3 minutes, then wiped away in a one-directional motion. These steps were repeated and then 2 swabs were taken from each square, processed and tested.

Positive readings=Monoplex>2000; Diplex>0

TABLE 75
Monoplex

| Swab # | GC | AC |
|---|---|---|
| 1 | 420 | 35365 |
| 2 | 395 | 35670 |
| 3 | 425 | 42341 |
| 4 | 435 | 34112 |
| 5 | 469 | 34857 |
| 6 | 509 | 28802 |
| 7 | 283 | 35825 |
| 8 | 508 | 42276 |
| 9 | 323 | 32138 |
| 10 | 392 | 32658 |
| 11 | 164 | 33870 |
| 12 | 173 | 31391 |
| 13 | 6571 | 33740 |
| 14 | 611 | 28683 |
| 15 | 1482 | 28890 |
| 16 | 624 | 33873 |
| 17 | 217 | 36900 |
| 18 | 665 | 34610 |
| 19 | 379 | 35350 |
| 20 | 567 | 34898 |
| 21 | 380 | 37342 |
| 22 | 658 | 32614 |
| 23 | 280 | 37890 |
| 24 | 589 | 36614 |

TABLE 76
Diplex

| Swab # | dGC | IAC |
|---|---|---|
| 1 | 31.9 | 40.8 |
| 2 | 14.6 | 35.6 |
| 3 | 0 | 36 |
| 4 | 0 | 41.3 |
| 5 | 27.3 | 38.9 |
| 6 | 0 | 39.9 |
| 7 | 0 | 39.9 |
| 8 | 0 | 41 |
| 9 | 0 | 42.3 |
| 10 | 0 | 38 |
| 11 | 0 | 38.9 |
| 12 | 0 | 43 |
| 13 | 0 | 40.8 |
| 14 | 0 | 37.8 |
| 15 | 0 | 37.6 |
| 16 | 0 | 39 |
| 17 | 0 | 44.1 |
| 18 | 31.5 | 41.7 |
| 19 | 27.1 | 43.2 |
| 20 | 0 | 40.6 |
| 21 | 0 | 39.5 |
| 22 | 0 | 43.1 |
| 23 | 0 | 40.8 |
| 24 | 0 | 42.9 |

TABLE 77
Monoplex

| Swab # | GC | AC |
|---|---|---|
| 25 | 345 | 32407 |
| 26 | 605 | 35031 |
| 27 | 563 | 49398 |
| 28 | 12896 | 33637 |
| 29 | 6213 | 33046 |
| 30 | 601 | 27879 |
| 31 | 502 | 22786 |
| 32 | 715 | 16557 |
| 33 | 510 | 33502 |
| 34 | 778 | 46897 |
| 35 | 456 | 36605 |
| 36 | 458 | 37934 |
| 37 | 505 | 34226 |
| 38 | 579 | 27248 |
| 39 | 265 | 26513 |
| 40 | 116 | 24731 |
| 41 | 555 | 19289 |
| 42 | 252 | 19429 |
| 43 | 223 | 22409 |
| 44 | 344 | 20635 |
| 45 | 240 | 20295 |
| 46 | 438 | 19267 |
| 47 | 821 | 17697 |
| 48 | 381 | 18837 |

TABLE 78
Diplex

| Swab # | dGC | IAC |
|---|---|---|
| 25 | 0 | 34 |
| 26 | 0 | 42 |
| 27 | 0 | 42.7 |
| 28 | 14.5 | 32.9 |
| 29 | 18.7 | 40.7 |
| 30 | 0 | 31.5 |
| 31 | 25.4 | 37.4 |
| 32 | 0 | 39.1 |
| 33 | 0 | 39.5 |
| 34 | 0 | 36.4 |
| 35 | 0 | 38.6 |
| 36 | 0 | 41.5 |
| 37 | 0 | 43.4 |
| 38 | 0 | 37.4 |
| 39 | 0 | 40.3 |
| 40 | 0 | 28.3 |
| 41 | 0 | 39.7 |
| 42 | 0 | 40.9 |
| 43 | 0 | 41.3 |
| 44 | 0 | 38.8 |
| 45 | 0 | 40.8 |
| 46 | 0 | 42.2 |
| 47 | 0 | 42.1 |
| 48 | 0 | 31.4 |

TABLE 79
Monoplex

| Swab # | GC | AC |
|---|---|---|
| 49 | 588 | 32098 |
| 50 | 631 | 39291 |
| 51 | 1441 | 40568 |
| 52 | 5856 | 42414 |
| 53 | 529 | 33702 |
| 54 | 490 | 44296 |
| 55 | 202 | 35479 |
| 56 | 433 | 55877 |
| 57 | 231 | 26525 |
| 58 | 496 | 34609 |
| 59 | 1073 | 29498 |
| 60 | 1045 | 47527 |
| 61 | 1290 | 38073 |
| 62 | 724 | 34092 |
| 63 | 939 | 34940 |
| 64 | 648 | 34841 |
| 65 | 404 | 39464 |
| 66 | 388 | 42125 |
| 67 | 565 | 44607 |
| 68 | 904 | 47893 |
| 69 | 656 | 35403 |
| 70 | 319 | 36662 |
| 71 | 10088 | 25822 |
| 72 | 483 | 17999 |

TABLE 80
Diplex

| Swab # | dGC | IAC |
|---|---|---|
| 49 | 0 | 37.8 |
| 50 | 0 | 40.6 |
| 51 | 0 | 38.7 |
| 52 | 0 | 38.6 |
| 53 | 0 | 36.5 |
| 54 | 0 | 36.2 |
| 55 | 0 | 34.6 |
| 56 | 0 | 24.9 |
| 57 | 0 | 38.7 |
| 58 | 0 | 41.2 |
| 59 | 1.4 | 40.8 |
| 60 | 0 | 39.7 |
| 61 | 0 | 41 |
| 62 | 0 | 38.9 |
| 63 | 0 | 40.2 |
| 64 | 0 | 41.7 |
| 65 | 11.2 | 35.6 |
| 66 | 0 | 32.6 |
| 67 | 0 | 34.2 |
| 68 | 0 | 33.3 |
| 69 | 0 | 36.6 |
| 70 | 0 | 34.7 |
| 71 | 0 | 36 |
| 72 | 26.3 | 32.7 |

TABLE 81
Monoplex

| Swab # | GC | AC |
|---|---|---|
| 73 | 807 | 42662 |
| 74 | 378 | 46540 |
| 75 | 917 | 46320 |
| 76 | 590 | 50889 |
| 77 | 703 | 32475 |
| 78 | 636 | 43325 |
| 79 | 269 | 47888 |
| 80 | 556 | 47134 |
| 81 | 409 | 36989 |
| 82 | 326 | 42705 |
| 83 | 622 | 44882 |
| 84 | 390 | 37163 |
| 85 | 573 | 33701 |
| 86 | 658 | 32927 |
| 87 | 609 | 43897 |
| 88 | 431 | 46131 |
| 89 | 13750 | 34084 |
| 90 | 442 | 35375 |
| 91 | 972 | 38919 |
| 92 | 435 | 39781 |
| 93 | 837 | 41371 |
| 94 | 309 | 48695 |
| 95 | 396 | 36321 |
| 96 | 366 | 31143 |

TABLE 82
Diplex

| Swab # | dGC | IAC |
|---|---|---|
| 73 | 0 | 38.4 |
| 74 | 0 | 20.6 |
| 75 | 0 | 34.2 |
| 76 | 16.1 | 35 |
| 77 | 0 | 38.7 |
| 78 | 0 | 38.3 |
| 79 | 0 | 38.2 |
| 80 | 0 | 35 |
| 81 | 0 | 38.9 |
| 82 | 0 | 38.7 |
| 83 | 0 | 39.1 |
| 84 | 0 | 35.5 |
| 85 | 0 | 36.6 |
| 86 | 0 | 36.7 |
| 87 | 0 | 37.3 |
| 88 | 0 | 35.7 |
| 89 | 0 | 38.5 |
| 90 | 12.4 | 37.9 |
| 91 | 0 | 41.3 |
| 92 | 0 | 39 |
| 93 | 4.2 | 37.2 |
| 94 | 0 | 37.3 |
| 95 | 0 | 35.3 |
| 96 | 0 | 35.6 |

We claim:

1. A method of reducing nucleic acid contamination on a surface comprising:
contacting the surface to be decontaminated with a solution consisting essentially of hydrogen peroxide and water; and
wiping the hydrogen peroxide and water solution from the surface.

2. The method of claim 1 further comprising the step of allowing the hydrogen peroxide and water solution to stand for at least about 3 minutes before wiping.

3. The method of claim 1 wherein the concentration of hydrogen peroxide in the solution is between about 0.5% and about 30%.

4. The method of claim 1 wherein the concentration of hydrogen peroxide in the solution is between about 2% and about 10%.

5. The method of claim 1 wherein the concentration of hydrogen peroxide in the solution is about 3%.

6. The method of claim 1, wherein the method comprises the additional step of rinsing the surface with water.

7. The method of claim 1, wherein the contamination comprises radioactive contaminants.

8. A method of reducing nucleic acid contamination on an item comprising:
soaking the item in a solution consisting essentially of hydrogen peroxide and water.

9. The method of claim 8, wherein the concentration of hydrogen peroxide in the solution is between about 0.5% and about 30%.

10. The method of claim 8, wherein the concentration of hydrogen peroxide in the solution is about 3%.

11. The method of claim 8, wherein the contamination comprises radioactive contaminants.

12. The method of claim 1, in which the contacting step is performed by soaking a towel or a wipe in a solution consisting essentially of hydrogen peroxide and water; and
wiping the surface to be cleaned with the soaked wipe or towel.

13. The method of claim 12, wherein the concentration of hydrogen peroxide in the solution is between about 0.5% and about 30%.

14. The method of claim 12, wherein the concentration of hydrogen peroxide in the solution is about 3%.

15. The method of claim 12, further comprising the step of wiping the surface with a dry towel or wipe.

16. The method of claim 13, further comprising the step of wiping the surface with a towel soaked with water.

17. The method of claim 13, further comprising the step of rinsing the surface with water.

18. A method of reducing nucleic acid contamination on a surface consisting of:
contacting the surface to be decontaminated with a solution consisting essentially of hydrogen peroxide and water; and
wiping the solution from the surface.

19. The method of claim 18, wherein the concentration of hydrogen peroxide in the solution is between about 0.5% and about 30%.

20. The method of claim 18, wherein the concentration of hydrogen peroxide in the solution is about 3%.
